(19) United States
(12) Patent Application Publication
(10) Pub. No.: US 2013/0237609 A1
(43) Pub. Date: Sep. 12, 2013

(54) CELLULOSE DERIVATIVES FOR ENHANCING BIOAVAILABILITY OF FLAVONOIDS

(71) Applicants: Kevin J. Edgar, Blacksburg, VA (US); Bin Li, Blacksburg, VA (US); Lynne Taylor, West Lafayette, IN (US); Grace Ilevbare, West Lafatette, IN (US); Stephanie M. Williams, Cleveland Heights, OH (US)

(72) Inventors: Kevin J. Edgar, Blacksburg, VA (US); Bin Li, Blacksburg, VA (US); Lynne Taylor, West Lafayette, IN (US); Grace Ilevbare, West Lafatette, IN (US); Stephanie M. Williams, Cleveland Heights, OH (US)

(21) Appl. No.: 13/880,521

(22) PCT Filed: Oct. 19, 2011
(86) PCT No.: PCT/US11/56946

§ 371 Date: Apr. 19, 2013

(51) Int. Cl. A61K 31/12

(52) U.S. Cl. CPC A61K 31/12
USPC 514/679

(57) ABSTRACT

The present invention relates to delivery systems to enhance the bioavailability of flavonoids to improve human health. Flavonoids of interest include but are not limited to curcumin, resveratrol, ellagic acid, naringenin, and quercetin. Flavonoids are important in part because they are known to have beneficial effects on human health, including cardioprotective, antioxidant, and anticancer effects. Utility has been limited by low bioavailability, both in the sense of requiring high doses and in that it has been difficult to carry out proper dose-response studies in the absence of methods to control the actual dose administered. In addition to pharmaceutical applications, there are potential nutraceutical uses, for example in supplements that might be sold in health food stores and pharmacies.

### CROSS-REFERENCE TO RELATED APPLICATIONS

This application relies on the disclosure of and claims the benefit of the filing date of U.S. Provisional Application No. 61/394,586, filed Oct. 19, 2010, the disclosure of which is incorporated by reference herein in its entirety.

### STATEMENT OF GOVERNMENT INTEREST

This invention was made under government support awarded by USDA/CSREES (now NIFA) under Grant No. 2009-35603-05068 and by the National Science Foundation under Grant No. DMR0804501.

### BACKGROUND OF THE INVENTION

1. Field of the Invention

The present invention relates to flavonoid compositions with high bioavailability. Embodiments of the invention include a solid dispersion of one or more flavonoids in a cellulose derivative matrix. Provided according to embodiments of the invention are flavonoids having enhanced solubility and chemical stability as compared with flavonoids alone or compared with physical mixtures of flavonoids with cellulose derivatives.

2. Description of Related Art

Flavonoids are known to have beneficial effects on human health, including cardioprotective, antioxidant, and anticancer effects. Curcumin, resveratrol, ellagic acid, naringenin, and quercetin are of interest for these purposes. Utility of flavonoids has been limited, however, by their low bioavailability and consequent need for high doses. Further, the absence of methods to control the actual dose administered makes it difficult to carry out proper dose-response studies for flavonoids.

Low bioavailability of certain drugs can be related to solubility of the drug. Solubility is often a critical issue in drug delivery in that the drug is not capable of permeating the epithelium and reaching the bloodstream if it is not dissolved in aqueous gastrointestinal lumen first. According to the Biopharmaceutics Classification System (BCS), BCS Class II type drugs are characterized by having high intestinal permeability but low solubility. It is understood that enhancing solubility of a BCS Class II compound almost invariably gives higher bioavailability. As oral drug delivery is preferred by patients and it is highly desirable to convert other delivery modes to oral, where possible, it would be highly desirable to enhance solubility of flavonoids to increase their bioavailability.

In recent years the flavonoid curcumin has received increasing attention due to its beneficial pharmacological properties including anti-oxidant, anti-cancer, anti-inflammatory, anti-carcinogenic, anti-bacterial, and anti-coagulant effects. Aggarwal, B. B.; Surh, Y.-J.; Shishodia, S., The molecular targets and therapeutic uses of curcumin in health and disease. ed.; Springer: New York, N.Y., 2007; and Kumar, A.; Ahuja, A.; Ali, J.; Baboota, S., Conundrum and therapeutic potential of curcumin in drug delivery. Crit. Rev Ther Drug Carrier Syst 2010, 27, (Copyright © 2011 U.S. National Library of Medicine.), 279-312.

Curcumin has been widely used as a natural yellow pigment in the food and textile industries. Phase I clinical trials confirmed the safety of curcumin doses up to 12 g daily, with no discernible toxicity other than mild nausea and diarrhea. Lao, C.; Ruffin, M.; Normolle, D.; Heath, D.; Murray, S.; Bailey, J.; Boggs, M.; Crowell, J.; Rock, C.; Brenner, D., Dose escalation of a curcuminoid formulation. BMC Complementary and Alternative Medicine 2006, 6, (1), 10; and Shoba, G., Influence of Piperine on the Pharmacokinetics of Curcumin in Animals and Human Volunteers. Planta medica 1998, 64, (04), 353-356 (“Shoba 1998”).

One drawback of curcumin, however, is its low bioavailability. Contributors to poor curcumin bioavailability include its poor aqueous solubility, its chemical instability, and its metabolic susceptibility. The maximum solubility of curcumin in aqueous buffer (pH 5.0) was reported to be only 11 ng/mL. Tonnesen, H. H.; Masson, M.; Loftsson, T., Studies of curcumin and curcuminoids. XXVII. Cyclodextrin complexation: solubility, chemical and photochemical stability, Int J Pharm 2002, 244, (1-2), 127-135. In addition, it was found that curcumin degraded very quickly in neutral or alkaline phosphate buffer solution, Wang, Y.-J.; Pan, M.-H.; Cheng, A.-L.; Lin, L.-I.; Ho, Y.-S.; Hsieh, C.-Y.; Lin, J.-K, Stability of curcumin in buffer solutions and characterization of its degradation products, Journal of Pharmaceutical and Biomedical Analysis 1997, 15, (12), 1867-1876; and Priyadarsini, K. I., Photophysics, photochemistry and photobiology of curcumin: Studies from organic solutions, bio-mimetics and living cells, J. Photoch Photobio C 2009, 10, (2), 81-95. Accordingly, drug delivery systems that would increase the solubility and chemical stability of curcumin are important targets to enable the use of curcumin for enhancing human health.

Since the “call to arms” to develop such enhanced delivery methods for biomedically-promising flavonoids in 2007, Hu, M., Commentary: Bioavailability of Flavonoids and Polyphenols: Call to Arms, Molecular Pharmaceutics 2007, 4, (6), 803-806, many researchers have sought to enhance the water solubility, stability and bioavailability of curcumin for preclinical and clinical research and application. Anand, P.; Kunnumakkara, A. B.; Newman, R. A.; Aggarwal, B. B., Bioavailability of Curcumin: Problems and Promises. Molecular Pharmaceutics 2007, 4, (6), 807-818.

Although alternative forms of curcumin administration have been explored (e.g., intravenous, transdermal), Patel, N.; Patel, R., Design and Evaluation of Transdermal Drug Delivery System for Curcumin as an Anti-Inflammatory Drug, Drug Dev Ind Pharm 2009, 35, (2), 234-242, enhancement of oral bioavailability of curcumin is still very important since oral administration is the most convenient and practical way to administer drugs, particularly those for daily or more frequent administration.

Researchers have recently investigated strategies including adjuvants (Shoba 1998), chemical modification (Wan, S. B.; Yang, H. J.; Zhou, Z. Y.; Cui, Q. C.; Chen, D.; Kanwar, J.; Mohammad, I.; Dou, Q. P.; Chan, T. H., Evaluation of curcumin acetates and amino acid conjugates as proteasome inhibitors, Int J Mol Med 2010, 26, (4), 447-455 (“Wan 2010”); and (“Tang 2010”), H. D.; Murphy, C. J.; Zhang, B.; Shen, Y. Q.; Van Kirk, E. A.; Murdoch, W. J.; Radosz, M., Curcumin polymers as anticancer conjugates, Biomaterials 2010, 31, (27), 7139-7149); and novel formulations such as nanoparticles, (Yen, F. L.; Wu, T. H.; Tzeng, C. W.; Lin, L. T.; Lin, C. C., Curcumin Nanoparticles Improve the Physicochemical Properties of Curcumin and Effectively Enhance Its Antioxidant and Antihepatoma Activities, J Agr Food Chem 2010, 58, (12), 7376-7382 (“Yen 2010”); and Mohanty, C.; Sahoo, S. K., The in vitro stability and in vivo pharmacokinetics of curcumin prepared as an aqueous nanoparticulate formulation, Biomaterials 2010, 31, (25), 6597-6611 (“Mohanty I 2010”)), micelles, (Yu, H. L.; Huang, Q. R., Enhanced in vitro anti-cancer activity of curcumin encapsulated in hydrophobically modified starch, Food Chem 2010, 119, (2), 669-674 (“Yu 2010”); and Mohanty, C.; Acharya, S.; Mohanty, A. K.; Dilnawaz, F.; Sahoo, S. K., Curcumin-encapsulated MePEG/PCL diblock copolymeric micelles: a novel controlled delivery vehicle for cancer therapy, Nanomedicine-Uk 2010, 5, (3), 433-449 (“Mohanty II 2010”)), cyclodextrin complexes, (Singh, R.; Tonnesen, H. H.; Vogensen, S. B.; Loftsson, T.; Masson, M., Studies of curcumin and curcuminoids, XXXVI, The stoichiometry and complexation constants of cyclodextrin complexes as determined by the phase-solubility method and UV-Vis titration, J Incl Phenom Macro 2010, 66, (3-4), 335-348 (“Singh 2010”); and Yadav, V. R.; Prasad, S.; Kannappan, R.; Ravindran, J.; Chaturvedi, M. M.; Vaahtera, L.; Parkkinen, J.; Aggarwal, B. B., Cyclodextrin-complexed curcumin exhibits anti-inflammatory and antiproliferative activities superior to those of curcumin through higher cellular uptake, Biochem Pharmacol 2010, 80, (7), 1021-1032 (“Yadav 2010”); and Yallapu, M. M.; Jaggi, M.; Chauhan, S. C., Poly(beta-cyclodextrin)/Curcumin Self-Assembly: A Novel Approach to Improve Curcumin Delivery and its Therapeutic Efficacy in Prostate Cancer Cells, Macromol Biosci 2010, 10, (10), 1141-1151 (“Yallapu 2010”)), microemulsions (Wu, X. M.; Xu, J. H.; Huang, X. W.; Wen, C. X. Self-microemulsifying drug delivery system improves curcumin dissolution and bioavailability. Drug Dev Ind Pharm 2011, 37, (1), 15-23), and solid dispersions (Phaechamud, T.; Sotanaphun, U., Dissolution of curcuminoids from solid dispersion using different carriers, Res. J. Pharm., Biol. Chem. Sci. 2010, 1, 198-206 (“Phaechamud 2010”); and Onoue, S.; Takahashi, H.; Kawabata, Y.; Seto, Y.; Hatanaka, J.; Timmermann, B.; Yamada, S., Formulation design and photochemical studies on nanocrystal solid dispersion of curcumin with improved oral bioavailability, J Pharm Sci 2010, 99, (Copyright © 2011 U.S. National Library of Medicine.), 1871-81 (“Onoue 2010”)).

Chemical modification of curcumin has been expiated to improve its bioavailability and biological activity. Wan 2010; Zeng, J.; Yang, N.; Li, X. M.; Shami, P. J.; Zhan, J. X., 4′-O-Methylglycosylation of Curcumin by Beauveria bassiana, Nat Prod Commun 2010, 5, (1), 77-80; and Sarkar, F. H.; Li, Y. W.; Wang, Z. W.; Padhye, S., Lesson Learned from Nature for the Development of Novel Anti-Cancer Agents. Implication of Isoflavone, Curcumin, and their Synthetic Analogs, Curr Pharm Design 2010, 16, (16), 1801-1812. A polyacetal based on curcumin was reported with higher antitumor activity. (Tang 2010).

Besides the above efforts, a number of studies approached aqueous curcumin solubility and stability improvement by reduction of curcumin particle size (micro- or nano-particle), or by stabilization by water soluble hosts (such as surfactants and cyclodextrin derivatives) or cellular components like liposomes to form micelles or complexes. (Yen 2010); (Mohanty I 2010); (Yu 2010); (Mohanty II 2010); (Singh 2010); (Yadav 2010); (Yallapu 2010); and Mazzarino, L.; Dora, C. L.; Bellettini, I. C.; Minatti, E.; Cardoso, S. G.; Lemos-Senna, E., Curcumin-Loaded Polymeric and Lipid Nanocapsules: Preparation, Characterization and Chemical Stability Evaluation. Lat Am J Pharm 2010, 29, (6), 933-940.

Amorphous solid dispersion is an important way to improve drug solubility and bioavailability for oral delivery; it has been known for approximately 40 years, but only recently has increased in interest as issues of formulation stability have been successfully addressed, Singh, M. C.; Sayyad, A. B.; Sawant, S. D., Review on various techniques of solubility enhancement of poorly soluble drugs with special emphasis on solid dispersion, Journal of pharmacy research 2010, 3, (10), 2494; Qian, F.; Huang, J.; Hussain, M. A., Drug-polymer solubility and miscibility: Stability consideration and practical challenges in amorphous solid dispersion development, J Pharm Sci-Us 2010, 99, (7), 2941-2947; Timpe, C., Drug solubilization strategies: applying nanoparticulate formulation and solid dispersion approaches in drug development, American pharmaceutical review 2010, 13, (1), 12; Tiwari, R., Solid dispersions: an overview to modify bioavailability of poorly water soluble drugs, International journal of pharmtech research 2009, 1, (4), 1338; an Leuner, C.; Dressman, J., Improving drug solubility for oral delivery using solid dispersions, European journal of pharmaceutics and biopharmaceutics 2000, 50, (1), 47-60 (Leuner 2000).

Drugs with a high degree of crystallinity often exhibit poor water solubility, since the lattice energy must be overcome in order for dissolution to occur. Molecular dispersion of the drug in a polymer matrix traps it in an amorphous solid state. Drug dissolution from this amorphous solid dispersion avoids the lattice energy barrier and thus may afford a supersaturated drug solution. These molecular drug-polymer dispersions may be prepared by co-extrusion, co-precipitation into a common non-solvent, spray-drying, freeze-drying, rotary evaporation, or film casting and subsequent drying. Certain polymers known to be safe for pharmaceutical use have received the most attention as amorphous dispersion polymers, including PEG, PVP, and certain polysaccharide derivatives like hydroxypropylmethylcellulose (HPMC) and hydroxypropylmethylcellulose acetate succinate (HPMCAS). (Leuner 2000).

Recently, HPMCAS and carboxymethyl cellulose acetate butyrate (CMCAB) have been shown to be effective matrices for improving the water solubility and bioavailability of poorly water-soluble drugs. Shelton, M. C.; Posey-Dowty, J. D.; Lingerfelt, L.; Kirk, S. K.; Klein, S.; Edgar, K. J., Enhanced dissolution of poorly soluble drugs from solid dispersions in carboxymethylcellulose acetate butyrate matrices, ACS symposium series 2009, 1017, (polysaccharide materials), 93; and Friesen, D. T.; Shanker, R.; Crew, M.; Smithey, D. T.; Curatolo, W. J.; Nightingale, J. A. S., Hydroxypropyl Methylcellulose Acetate Succinate-Based Spray-Dried Dispersions: An Overview. Molecular Pharmaceutics 2008, 5, (6), 1003-1019.

Several authors have reported the solubility enhancement of curcumin by solid dispersion. (Phaechamud 2010); (Onoue 2010); Paradkar, A.; Ambike, A. A.; Jadhav, B. K.; Mahadik, K. R., Characterization of curcumin-PVP solid dispersion obtained by spray drying. Int J Pharm 2004, 271, (Copyright © 2011 U.S. National Library of Medicine. (“Pradakar 2004”)), 281-6; and Kaewnopparat, N.; Kaewnopparat, S.; Jangwang, A.; Maneenaun, D.; Chuchome, T.; Panichayupakaranant, P., Increased Solubility, Dissolution and Physicochemical Studies of Curcumin-Polyvinylpyrrolidone K-30 Solid Dispersions, World Academy of Science, Engineering and Technology 2009, 55, 229-234 (“Kaewnopparat 2009”).

Suresh et al. reported that curcumin could be solubilized using Cremophor and PEG 20000 to achieve a curcumin concentration of 18 mg/mL. Suresh, S.; Prasad, K. Solid dispersions of curcumin, Indian J. Pharm. Sci. 1999, 61, (Copyright © 2011 American Chemical Society (ACS).), 131-133. Onoue and co-workers reported formulation of curcumin as nanocrystals, as a solid dispersion in HPMCAS, and as a nanoemulsion. They observed solubility enhancement and improvement of oral bioavailability of curcumin up to 9-16 fold by these methods, including 12-fold enhancement by solid dispersion with HPMCAS at a 1:4 curcumin:HPMCAS ratio, prepared by freeze-drying (the impacts of choice of cellulose derivative or flavonoid:polymer ratio were not examined). (Onoue 2010).

Spray-dried curcumin/PVP solid dispersions were studied by Paradkar and Kaewnopparat, respectively. (Pradakar 2004); (Kaewnopparat 2009). The results indicated that Cur:PVP may form amorphous solid dispersions in ratios ranging from 1:8 to 1:2, and that both Cur solubility and dissolution rate from Cur/PVP solid dispersions were much greater than those from physical Cur:PVP mixtures or pure curcumin (Kaewnopparat 2009).

From the above discussion it is apparent that what is needed are compositions for increasing solubility of drugs, and in particular flavonoids, to provide for higher bioavailability of the drugs in oral form. Physical blends of amorphous drugs with cellulose derivatives can give pH-triggered, slow, often zero-order drug release that is well-suited to once-a-day pills for enhanced compliance. Molecular blends on the other hand in many cases afford largely or entirely amorphous drug intimately blended with cellulose derivatives, such as CMCAB. Release from these molecular blends seems to be rapid for water-soluble drugs, but nearly zero-order for otherwise poorly soluble drugs. Although existing art has found ways of increasing solubility of flavonoids, none provide for a composition with increased solubility, increased chemical stability, and increased bioavailability of flavonoids for oral delivery.

### SUMMARY OF THE INVENTION

The wide implementation of high throughput screening for potential therapeutic entities by the pharmaceutical industry has dramatically raised the number of poorly soluble drug candidates. As a result, the improvement of solubility and dissolution rate of poorly soluble compounds is of great importance.

Efficiencies in the drug development process can be gained by requests for waiver of in vivo bioavailability (BA) and/or bioequivalence (BE) studies for immediate release (IR) solid oral dosage forms when applying to the Food and Drug Administration (FDA) for approval of new drug applications (NDA), abbreviated new drug applications (ANDA), or investigational new drug (IND) applications. Such biowaivers can be requested for IR solid oral dosage forms based on an approach termed the Biopharmaceutics Classification System (BCS). “Waiver of In-vivo Bioavailability and Bioequivalence Studies for Immediate Release Solid Oral Dosage Forms Based on a Biopharmaceutics Classification System,” US Dept. of Health and Human Services, FDA, Center for Drug Evaluation and Research, August 2000, the disclosure of which is incorporated by reference herein in its entirety. Very generally, the BCS allows pharmaceutical companies to forego clinical bioequivalence studies, if their drug product meets the specification detailed in the guidance. A waiver of In-vivo Bioavailability and Bioequivalence studies based on the BCS classification can save pharmaceutical companies a significant amount of development time and reduce development costs.

The Biopharmaceutics Classification System (BCS) is a scientific framework for classifying drug substances based on their aqueous solubility and intestinal permeability. When combined with the dissolution of the drug product, the BCS takes into account three major factors that govern the rate and extent of drug absorption from IR solid oral dosage forms: dissolution, solubility, and intestinal permeability. According to the BCS, drug substances are classified as follows: Class 1: High Solubility-High Permeability; Class 2: Low Solubility-High Permeability; Class 3: High Solubility-Low Permeability; Class 4: Low Solubility-Low Permeability. In addition, IR solid oral dosage forms are categorized as having rapid or slow dissolution. Within this framework, when certain criteria are met, the BCS can be used as a drug development tool to help sponsors justify requests for biowaivers.

The inventors have found that by combining flavonoids in molecular blends with cellulose derivatives, drug solubility, stability, and/or bioavailability can be enhanced. Indeed, the amount of flavonoid released from administration of such compositions is approximately the amount of amorphous drug present. Specifically, the inventors have found that by delivering one or more flavonoids in amorphous solid form dispersed within an amorphous polymer matrix, the solubility and bioavailability of flavonoids can be enhanced. See FIG. 1.

The term molecular dispersion, and equivalents, in the context of this disclosure refers to a composition in which the dispersed phase either is not crystalline, or has crystalline domain sizes so small that they cannot be observed by commonly used techniques including XRD and DSC; these domains may approach the size of individual molecules. Molecular dispersion can be further categorized into 1) a solid solution, which refers to drug molecules dissolved or molecularly dispersed throughout the carrier polymer matrix in amorphous form (i.e., having no or negligible crystallization; and 2) a solid dispersion, which describes systems containing dispersed particles which can be amorphous or crystalline.

Embodiments of the invention provide flavonoid compositions having higher bioavailability than existing flavonoids and flavonoid compositions. Oral delivery to patients of the inventive flavonoid compositions may have one or more of the following advantages: a lower dose, lower cost, fewer side effects, lower variability between patients and within patients (dosage time, fed/fasted), and smaller dosage forms.

Even further, the inventors have found that by preparing a homogeneous mixture of a flavonoid compound and a carboxylated cellulose ester matrix, there is remarkable success at suppressing the crystallinity of even these high melting, highly crystalline flavonoids. These amorphous dispersions of flavonoid in carboxylated cellulose ester matrix, upon exposure to water or to aqueous buffer that mimics physiological fluid (pH 6.8 for the small intestine, pH 7.4 for plasma/blood, or pH 1.2 for stomach) afford dramatic solubility improvements for the flavonoid compound. These dramatic solubility improvements can lead to enhanced bioavailability, as they will enhance the concentration difference for the flavonoid across the biological membrane.

For example, these amorphous dispersions of flavonoid in carboxylated cellulose ester matrix, upon oral administration, can dissolve to create a much higher concentration of flavonoid in the gastrointestinal lumen in the small intestine, compared to that which would result from crystalline flavonoid alone. This creates a high concentration of flavonoid on the lumen side and a low concentration on the blood side, creating a strong driving force for flavonoid permeation into the blood.

Accordingly, one object of the present invention includes a composition comprising a molecular dispersion of an amorphous solid compound in an amorphous polymer matrix which has increased solubility, stability, or bioavailability over a non-amorphous form of the compound. Such compositions can include that the non-amorphous form of the compound is a Class II drug according to the Biopharmaceutics Classification System in that it exhibits high intestinal permeability but low solubility, or a Class IV drug characterized by low solubility and low permeability. Indeed, any drug with low solubility can have increased bioavailability by incorporating it into the polymer matrix of the invention, which enhances its solubility. Even further, the compositions can be such that the amorphous solid is chosen from at least one flavonoid chosen from curcumin, resveratrol, ellagic acid, naringenin, and quercetin.

Compositions of the invention also include compositions in which an amorphous polymer matrix is chosen from at least one of CA adipate, CAB adipate, CAP suberate, CAP sebacate, CAB suberate, CAB sebacate, CA suberate, CA sebacate, HPMCAS (hydroxypropylmethyl cellulose acetate succinate), CAPH (cellulose acetate phthalate), HPMCPH (hydroxypropylmethylcellulose phthalate), CAAdP (cellulose adipate ester), CMCAB (carboxymethylcellulose acetate butyrate), and PVP (polyvinylpyrrolidone).

In any composition of the invention, the amorphous polymer matrix can be chosen from at least one of carboxylated cellulose esters. Preferred carboxylated cellulose esters can be chosen from cellulose acetate adipate propionate, cellulose acetate adipate butyrate, cellulose acetate adipate, cellulose acetate propionate suberate, cellulose acetate propionate sebacate, carboxymethylcellulose acetate butyrate, carboxymethylcellulose acetate propionate, and hydroxymethylcellulose acetate succinate.

In embodiment, the amorphous solid compound and the amorphous polymer matrix are combined in a ratio of from about 1:100 to 100:1. For example, the compositions can be formulated such that a ratio of the amorphous solid compound to the amorphous polymer matrix is about 20:80 to about 80:20, or about 50:50, or about 40:60 to about 60:40, or about 25:75 to about 75:25, or about 1:9, 1:3, 1:1, 3:1, or 9:1.

Embodiments of the present invention also include pharmaceutical formulations comprising an amorphous solid form of a Class II drug according to the Biopharmaceutics Classification System, which drug exhibits high intestinal permeability but low solubility, wherein the amorphous solid is present in a molecular dispersion with an amorphous polymer matrix and the formulation has increased solubility, stability, or bioavailability over the Class II drug. For example, the pharmaceutical formulations can be such that the drug is a flavonoid chosen from curcumin, resveratrol, ellagic acid, naringenin, and quercetin.

In pharmaceutical formulations according to the invention, the amorphous polymer matrix can be at least one of HPMCAS (hydroxypropylmethyl cellulose acetate succinate), CAPH (cellulose acetate phthalate), HPMCPH (hydroxypropylmethylcellulose phthalate), CAAdP (cellulose adipate ester), CMCAB (carboxymethylcellulose acetate butyrate), PVP (polyvinylpyrrolidone), and PEG (polyethylene glycol).

Further, the pharmaceutical formulations can comprise an amorphous polymer matrix chosen from at least one of carboxylated cellulose esters. Preferred carboxylated cellulose esters can be chosen from cellulose acetate adipate propionate, cellulose acetate adipate butyrate, cellulose acetate adipate, cellulose acetate propionate suberate, cellulose acetate propionate sebacate, carboxymethylcellulose acetate butyrate, carboxymethylcellulose acetate propionate, and hydroxymethylcellulose acetate succinate.

Pharmaceutical formulations of the invention can comprise the amorphous solid compound and the amorphous polymer matrix in a ratio of from about 1:10 to 10:1. For example, the formulations can comprise such a ratio of the amorphous solid compound to the amorphous polymer matrix that is about 1:9, 1:3, 1:1, 3:1, or 9:1.

Also provided, is a method of treating or ameliorating a disease by providing cardioprotective, antioxidant, or anticancer effects by administering to a patient in need thereof a pharmaceutical formulation comprising a flavonoid chosen from curcumin, resveratrol, ellagic acid, naringenin, and quercetin in a polymer matrix. Such methods of treating can employ any composition or pharmaceutical formulation disclosed or suggested in this disclosure and one of ordinary skill in the art will know which compositions and formulations would be applicable for treating specific diseases.

Likewise, provided is a method for the preparation of a medicament for treating or ameliorating a disease responsive to cardioprotective, antioxidant, or anticancer agents, wherein the medicament comprises a flavonoid chosen from curcumin, resveratrol, ellagic acid, naringenin, and quercetin in a polymer matrix. Medicaments can include any composition and/or pharmaceutical formulation described or suggested in this disclosure.

Methods for manufacturing the compositions, pharmaceutical formulations, and medicaments of embodiments of the invention are also included in the scope of this disclosure.

The features of novelty and various other advantages that characterize the invention are pointed out with particularity in the claims forming a part hereof. However, for a better understanding of the invention, its advantages, and the objects obtained by its use, reference should be made to the drawings that form a further part hereof, and to the accompanying descriptive matter, in that there is illustrated and described a preferred embodiment of the invention. The features and advantages of the present invention will be apparent to those skilled in the art. While numerous changes may be made by those skilled in the art, such changes are within the spirit of the invention.

### BRIEF DESCRIPTION OF THE DRAWINGS

These drawings illustrate certain aspects of some of the embodiments of the present invention, and should not be used to limit or define the invention.

FIG. 1 is schematic diagram representing a molecular dispersion of a drug in a polymer matrix, e.g., CMCAB matrix.

FIGS. 2A-E are structural formulas of flavonoid compounds in embodiments of the invention, such as curcumin, quercetin, ellagic acid, naringenin, and resveratrol.

FIGS. 3A-E are structural formulas of polymers that may be used in embodiments of the invention, including PVP, CMCAB, HPMCAS, CAPH, HPMCP, and CAAdP.

FIG. 4 is a graph showing release of a drug of the invention, griseofulvin, which is provided in a polymer matrix, such as a CMCAB matrix, HPMCAS matrix, or PVP matrix.

FIGS. 5A-B are XRD analyses of quercetin/CMCAB blends, comparing physical mixtures with that of spray-dried molecular blends.

FIG. 6 is an XRD analysis of quercetin in a spray-dried molecular blend within a CAPH, CMCAB, HPMCPH, PVP, or HPMCAS matrix.

FIGS. 7A-B provides XRD patterns of (A) Cur/CMCAB physical mixtures and (B) spray dried solid dispersions.

FIG. 8A is an XRD analysis of ellagic acid and ellagic acid spray-dried solid dispersion in a HPMCAS, CAPH, CMCAB, PVP, or HPMCPH polymer matrix.

FIG. 8B is an XRC analysis of naringenin and naringenin spray-dried solid dispersion in a HPMCAS or CMCAB matrix.

FIG. 9 is an XRD analysis of resveratrol/CMCAB spray dried blends.

FIG. 10 an XRD analysis of resveratrol/CAAdP spray dried blends.

FIGS. 11A-B are graphs showing (A) DSC heating curves of Cur, HPMCAS, Cur/HPMCAS physical mixtures and solid dispersions; and (B) DSC heating curves of Cur, CMCAB, Cur/CMCAB physical mixtures and solid dispersions.

FIG. 12 is a graph showing stability of curcumin in a CMCAB or HPMCAS matrix.

FIG. 13 is a graph showing T_{g} values of curcumin solid dispersions plotted against curcumin content.

FIGS. 14A-B are FTIR spectra of respectively: (A) FTIR spectra of Cur spray dried dispersions; and (B) FTIR spectra of 1:1 Cur/HPMCAS spray dried dispersion and physical mixture, Cur, and HPMCAS.

FIGS. 15A-B are NMR spectra of curcumin and a Cur/HPMCAS spray-dried blend.

FIGS. 16A-B are graphs showing the solubility of curcumin in various solvents.

FIGS. 17A-B are HPLC chromatograms comparing the stability of curcumin in pH 7.4 buffer for 24 h, which shows degradation products such as vanillin, with that of a curcumin/CMCAB spray-dried blend in pH 7.4 buffer for 24 h with reduced degradation.

FIG. 18 is a graph showing stability of Cur and Cur/CMCAB 1:9 solid dispersions in pH 7.4 buffer (HPLC after EtOH dilution).

FIGS. 19A-B are graphs showing stability of Cur and Cur/polymer solid dispersions in pH 7.4 (A) and 6.8 (B) buffer (UV-Vis after EtOH dilution).

FIGS. 20A-B are graphs showing stability of curcumin and Cur/polymer solid dispersions in pH 7.4 (A) and 6.8 (B) buffer (UV-Vis, no EtOH added).

FIGS. 21A-B are graphs showing the maximum Cur concentration from Cur/CMCAB (A) and Cur/HPMCAS (B) solid dispersions at pH 6.8.

FIG. 22 is a graph showing dissolution from Cur/HPMCAS solid dispersions (pH 6.8; AC=after centrifugation).

FIGS. 23A-B are graphs showing dissolution data for curcumin/HPMCAS blends.

FIG. 24 is a graph showing dissolution of Cur and Cur physical mixture and spray-dried blends with polymers (pH 6.8, UV-Vis).

FIG. 25 is a graph showing dissolution of Cur and Cur in spray-dried blends with polymers (pH 1.2, UV-Vis).

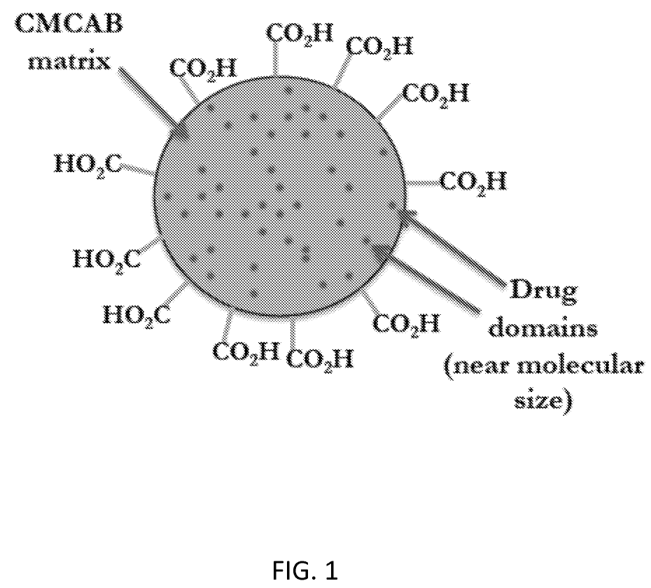

### DETAILED DESCRIPTION OF PREFERRED EMBODIMENTS

The present invention relates to compositions comprising one or more flavonoids in amorphous solid form dispersed by molecular dispersion within an amorphous polymer matrix. Molecular amorphous dispersions of flavonoids in cellulose ester are especially preferred, which have strongly enhanced solubility. Methods of making the molecular dispersion compositions as well as methods of using them are also included in embodiments of the invention. Compositions and methods of embodiments of the invention provide an effective and practical way to deliver flavonoids as dietary supplements or therapeutic compounds.

Flavonoids are water insoluble polyphenolic molecules, which are natural materials present in the plant world and in human diets, or are derived semisynthetically from those materials, and contain or are derived from the flavone moiety. They contain aromatic rings, carbonyl groups, and usually hydroxyl groups. They tend to be symmetrical and highly crystalline. The flavonoids consist of 6 major subgroups: chalcone, flavone, flavonol, flavanone, anthocyanins and isoflavonoids. Flavonoids are found in fruits, vegetables, and certain beverages that have diverse beneficial biochemical and antioxidant effects. For example, sources of flavonoids include: apples, pears, cabbage, raspberries, blueberries, parsley, and tomatoes to name a few. USDA Database for the Flavonoid Content of Selected Foods, Release 3, September 2011, the entire disclosure of which is incorporated by reference herein. Although any flavonoid can be used in this invention, even if not described herein, this disclosure provides for examples using current flavonoids of interest.

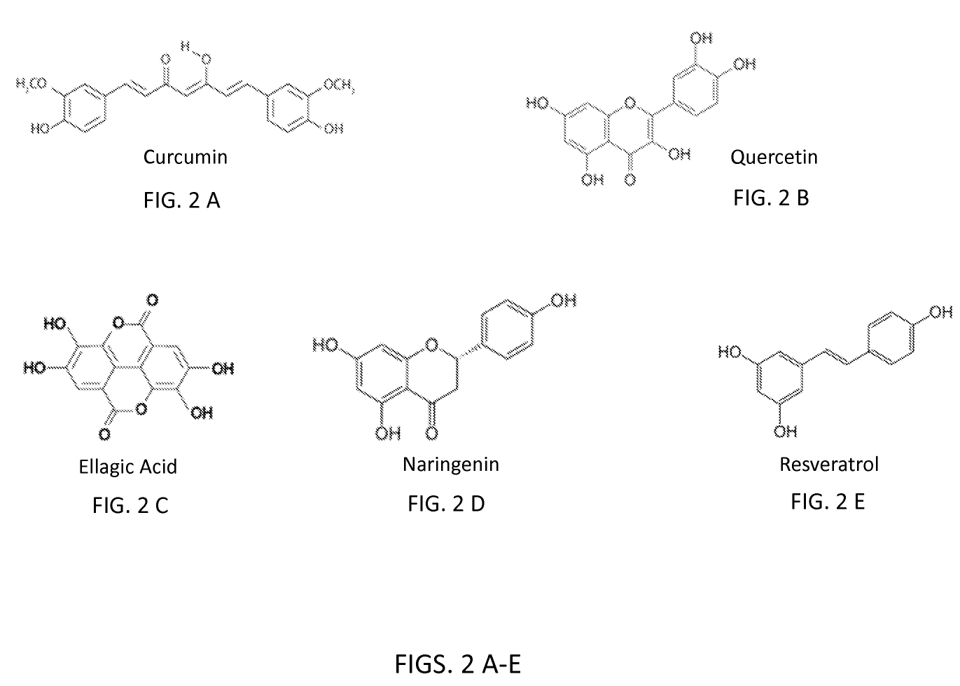

Specific flavonoids of interest that can be used in embodiments of this invention include but are not limited to curcumin, resveratrol, ellagic acid, naringenin, and quercetin. See FIGS. 2A-E. Representative sources of these beneficial phytochemicals are shown below in Table I, as well as some of their corresponding health benefits, solubility, and melting points.

TABLE I
Representative Flavonoids

| | | | | | MP |
|---|---|---|---|---|---|
| Flavonoid | Sources | Example Activity | Solubility | | (° C.) |
| Curcumin | Turmeric | Anti-inflammatory | 3 | μg/mL | 183 |
| Ellagic Acid | Nuts, | Anti-oxidant | 10 | μg/mL | >360 |
| | berries | | | | |
| Naringenin | Grapefruit | Anti-ox, hepato- | 40 | μg/mL | 247-250 |
| | | protective | | | |
| Resveratrol | Red wine | Cardioprotective | <0.1 | μg/mL | 253-255 |
| Quercetin | Onions | Anti-cancer | 0.4 | μg/mL | >300 |

In particular, the inventors have found that curcumin appears to be an ideal substrate for solubility enhancement by molecular dispersion in solid polymer matrices, since it is only moderately hydrophobic (log P 2.5), but it has a high melting point (180° C.) Accordingly, preferred compositions and methods of the invention may comprise the flavonoid curcumin.

The polymer for providing the amorphous polymer matrix is selected such that it is preferably not absorbed by the body and is preferably not toxic, including its chemical or enzymatic breakdown by-products, if any. Additionally, the miscibility of the polymer is such that polymer-drug interactions (for example, CO₂H—:NR₃) are maximized and the polymer retains the ability to disperse in water.

Functions of the polymer include that it acts to stabilize the drug in supersaturated aqueous solution and minimizes, prolongs the onset of, or avoids or prevents crystallization of the drug. Another preferred characteristic of the polymer is that it may have a high Tg to immobilize drug against crystallization, even in the presence of high humidity (Pz) and high ambient temperature (50-60° C.) for years. Ideally, the polymer itself is amorphous. Release properties of the drug in such polymers can include pH control, slow release (ideally zero order), and/or once a day dosage or even less frequent dosage.

There are three key polymer performance characteristics for molecular dispersion formulations, listed here along with some of the key polymer properties that may provide those performance characteristics: 1) the ability to stabilize the drug against crystallization in the solid phase (similar solubility parameter to that of drug, specific polymer-drug interactions, high glass transition temperature (T_{g})); 2) the ability to stabilize the drug in solution after release but prior to absorption from the GI tract (at least slight (μg/mL) polymer solubility in pH 6.8 buffer, plus affinity for drug as in 1)); 3) the desired drug release profile (release rate will decline as polymer hydrophobicity rises, groups ionizable at neutral pH (e.g., —CO₂H) can provide release trigger). Properly designed carboxylated polysaccharide derivatives are excellent candidates for amorphous dispersion polymers, since as a class they tend to have low toxicity and high T_{g} values. A high T_{g} helps maintain the matrix in the glassy state at high humidity and relatively high ambient temperatures, in order to limit molecular motion of drug molecules and thus inhibit drug crystallization in storage and transport.

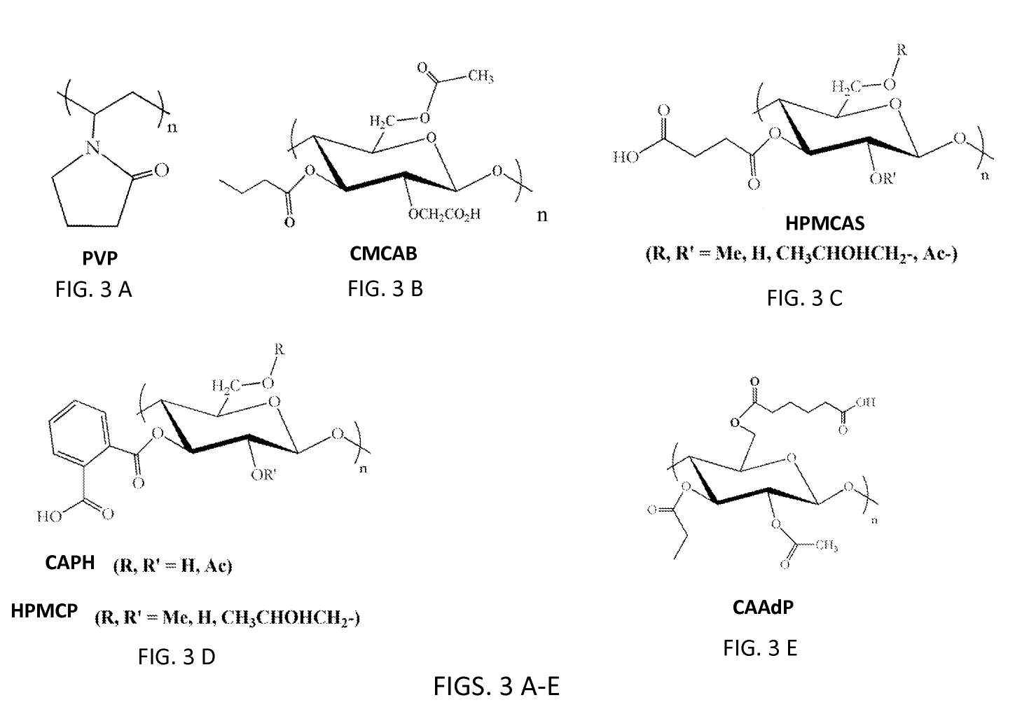

Amorphous dispersion polymers according to embodiments of the invention can include, but are not limited to, commercially available carboxylated cellulose derivatives, such as: HPMCAS (hydroxypropylmethyl cellulose acetate succinate), CAPH (cellulose acetate phthalate), HPMCPH (hydroxypropylmethylcellulose phthalate), CAAdP (cellulose adipate ester), and CMCAB (carboxymethylcellulose acetate butyrate). PVP (polyvinylpyrrolidone) and PEG (polyethylene glycol) can also be used. It has been found that molecular blends formed from cellulose adipates have enhanced hydrolytic stability as compared with succinate and glutarate. Non-limiting representative polymers that can be used in embodiments of the invention include, but are not limited to those shown in FIGS. 3A-E.

Cellulose polymers of this invention can be synthesized from cellulose, cellulose esters, or cellulose esters.

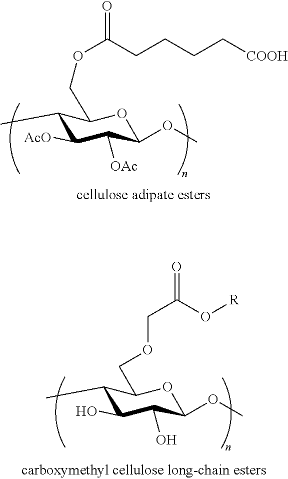

For example, carboxymethylcelulose acetate butyrate (CMCAB) can be synthesized according to the following scheme:

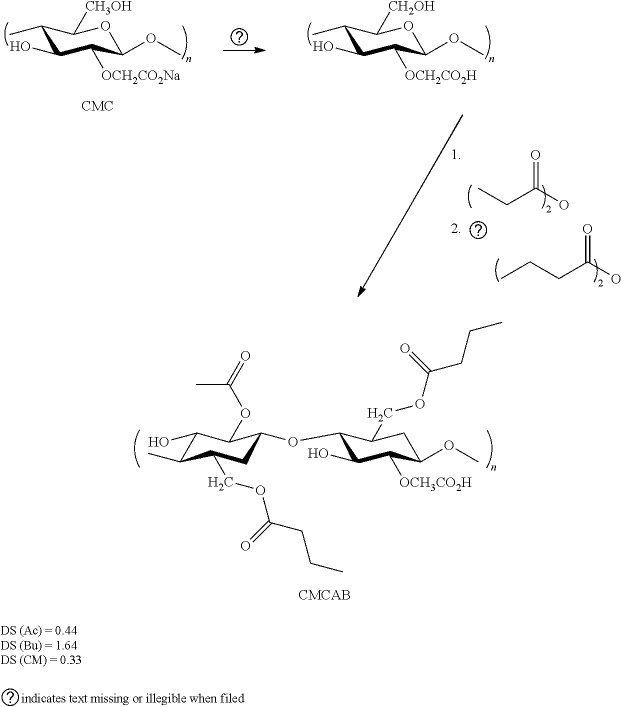

Preferred carboxylated cellulose esters of utility in this invention include but are not limited to cellulose acetate adipate propionate, cellulose acetate adipate butyrate, cellulose acetate adipate, cellulose acetate propionate suberate, cellulose acetate propionate sebacate, carboxymethylcellulose acetate butyrate, carboxymethylcellulose acetate propionate, and hydroxymethylcellulose acetate succinate. Preferred are cellulose esters that are relatively hydrophobic polymers, which aids their interaction with hydrophobic flavonoids and stabilizes the amorphous drug. It also slows the penetration of water into the matrix, and of aqueous drug solution back out of the matrix, thereby promoting desirable slow drug release. The carboxyl groups not only provide specific interactions with the flavonoid molecule to enhance stability of the amorphous flavonoid dispersion, but they provide the mechanism for drug release. Ionization of the carboxyl groups in the neutral pH of the small intestine causes swelling and/or dissolution of the carboxylated cellulose ester matrix, permitting an infusion of water into the matrix and thus drug dissolution.

The inventors have observed very dramatic solubility enhancements for flavonoids from such matrices. In addition, it is necessary to stabilize the aqueous flavonoid solution, after dissolution of the flavonoid from the carboxylated cellulose ester matrix, against flavonoid recrystallization from the resulting supersaturated flavonoid solution. Direct evidence of such stabilization has been acquired by the inventors. Ellagic acid, for example, is poorly soluble in organic solvents like acetone/ethanol. Addition of carboxymethylcellulose acetate butyrate to acetone/ethanol permitted dissolution of ellagic acid in this solution, where in the absence of carboxymethylcellulose acetate butyrate, no dissolution occurred.

Although an advantage of PVP is that it is water soluble, it has been found that PVP may be less effective than other polymer selections at preventing crystallization from solution. Although this characteristic does not eliminate PVP as a possible drug delivery vehicle, this may limit applicability of PVP in some applications. PEG is also water soluble, but prone to crystallize. HPMCAS is somewhat hydrophobic, has a pH release trigger, and tends to provide faster release of the drug due to its greater hydrophilicity. HPMCAS and CMCAB stabilize curcumin in solution. CMCAB and CAAdP are preferred polymers with regard to their ability to stabilize active molecules in solution, including flavonoids.

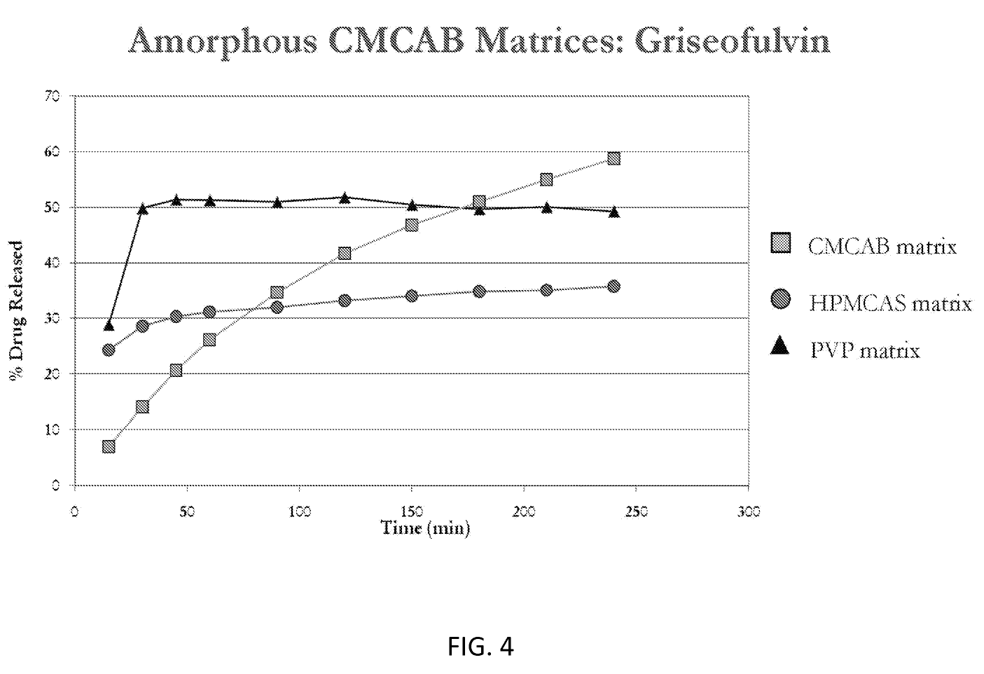

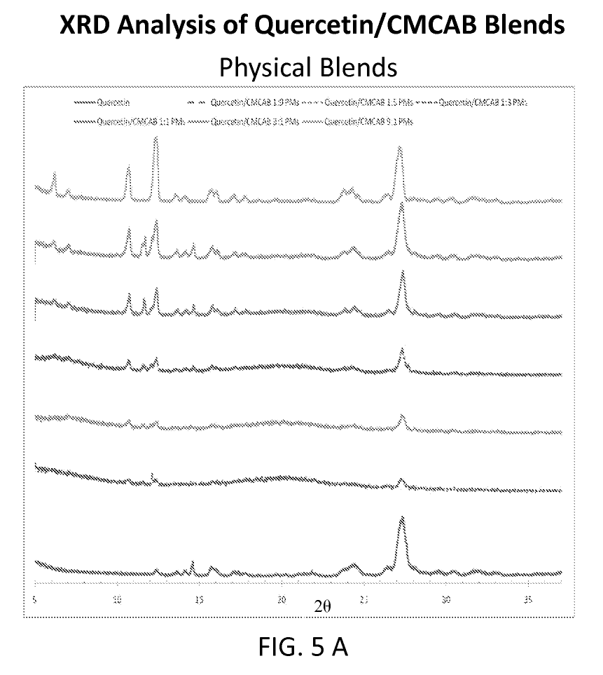

Indeed, comparative studies show that CMCAB is one of the more effective amorphous matrix polymers. See FIG. 4. As shown, bioavailability is increased with CMCAB as the matrix polymer, as compared with HPMCAS or PVP. In the polymer matrix the solubility of the antifungal griseofulvin is enhanced by up to about 2500 times that of crystalline griseofulvin (e.g., from 12 μg/mL up to 30 mg/mL). Griseofulvin is shown below in Formula I.

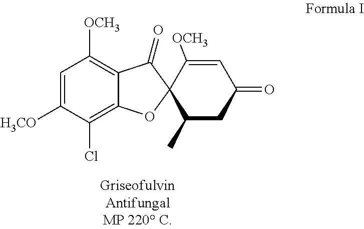

In preferred embodiments according to the invention, a molecular dispersion combining any one or more flavonoid chosen from quercetin, curcumin, resveratrol, naringenin, and ellagic acid with any one or more cellulose derivative chosen from CAAdP, CMCAB, HPMCAS, CAPH, or HPMCPH can be prepared in ratios of w:w of about 1:9, 1:3, 1:1, 3:1, or 9:1. Even further preferred are such compositions prepared by spray drying in a solvent of acetone:ethanol of about 1:4 with 2 wt % solids, under N₂ at about 50-90° C. A Buchi Mini-Spray Dryer B-290 is a preferred tool for preparing such compositions. Preferred conditions can include: a feed flow of about 9 ml/min., an N₂ flow of about 357 L/hr., an inlet temperature of about 90° C., an outlet temperature of about 57-62° C., and aspirator at 100%.

The resulting compositions can be analyzed by any number of qualitative or quantitative techniques to determine whether the composition is amorphous. Such techniques include analysis by X-Ray Diffraction (XRD) or Differential Scanning calorimetry (DSC) to determine crystallinity.

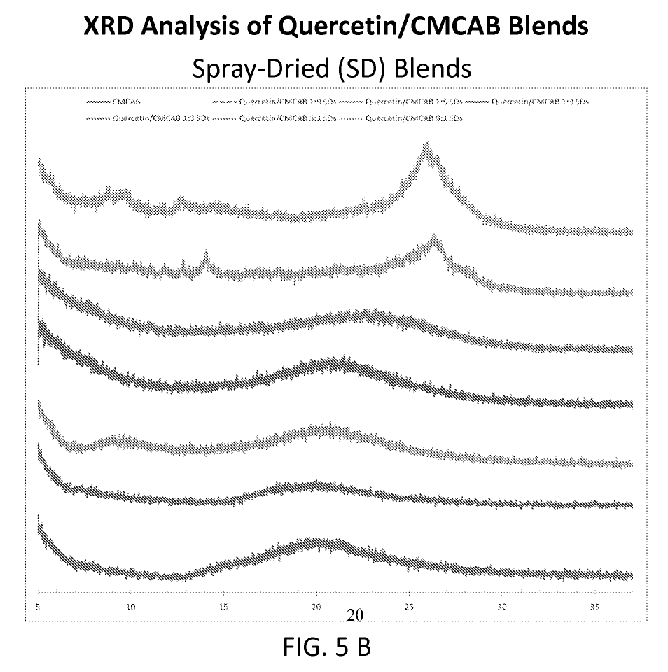

As shown in FIGS. 5A-B, XRD indicates quercetin is totally amorphous in the spray-dried samples up to about 50% flavonoid concentration in CMCAB.

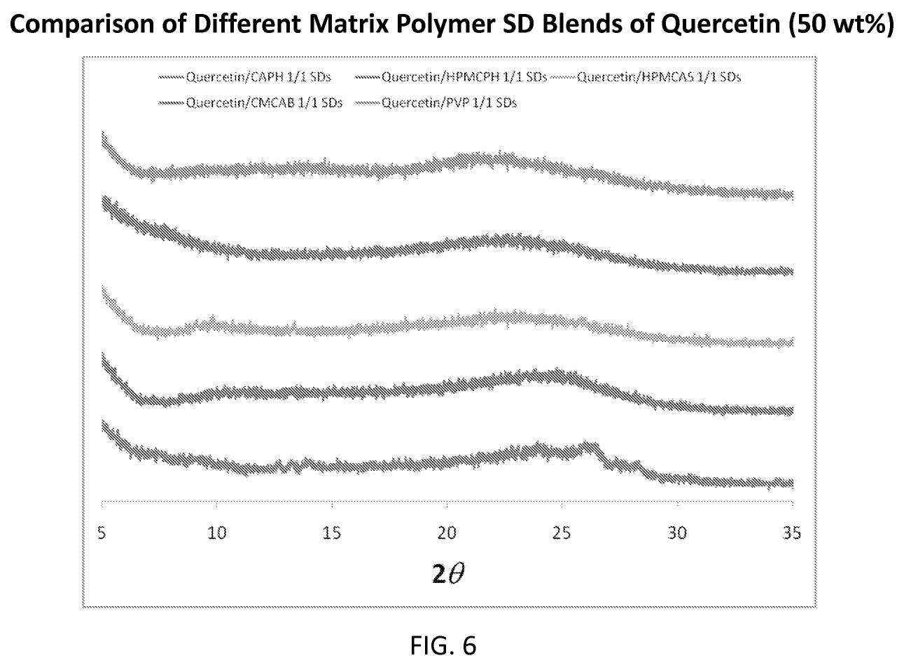

As shown in FIG. 6, quercetin is amorphous in spray dried molecular blends up to about 50% in CMCAB, HPMCAS, HPMCPH, and PVP, but only partly amorphous in up to about 50% in a CAPH blend.

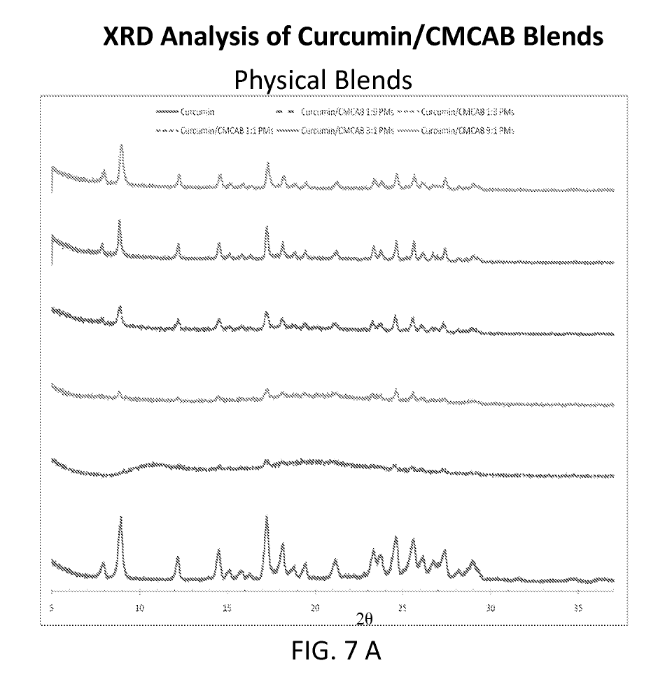

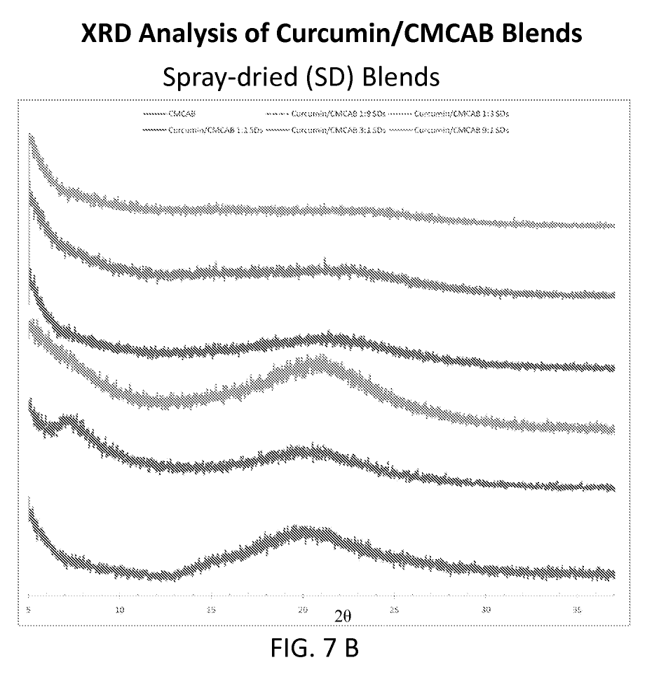

Similarly, FIGS. 7A-B show that curcumin is totally amorphous in molecular dispersion samples up to about a concentration of 90% in CMCAB or HPMCAS matrices and amorphous up to about 50% concentration in other polymer matrices.

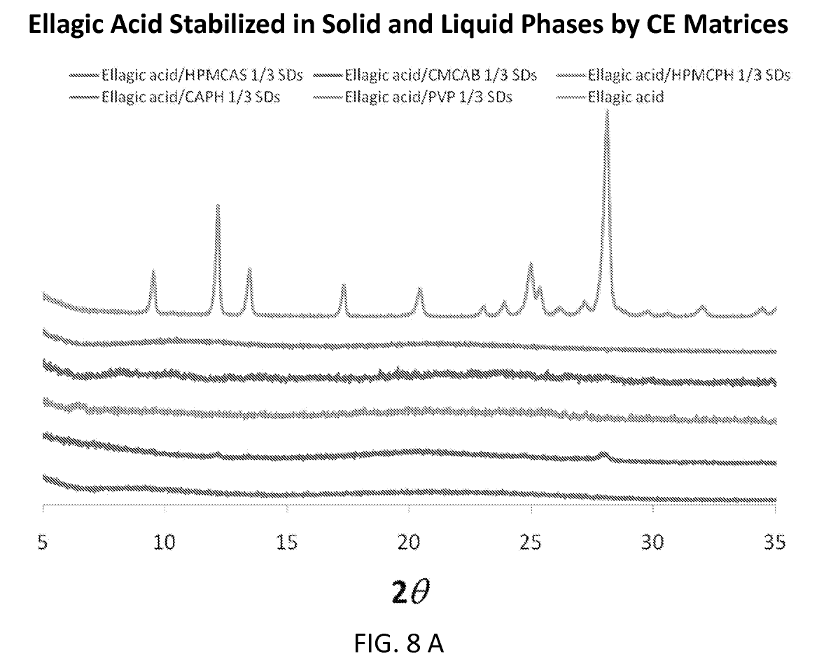

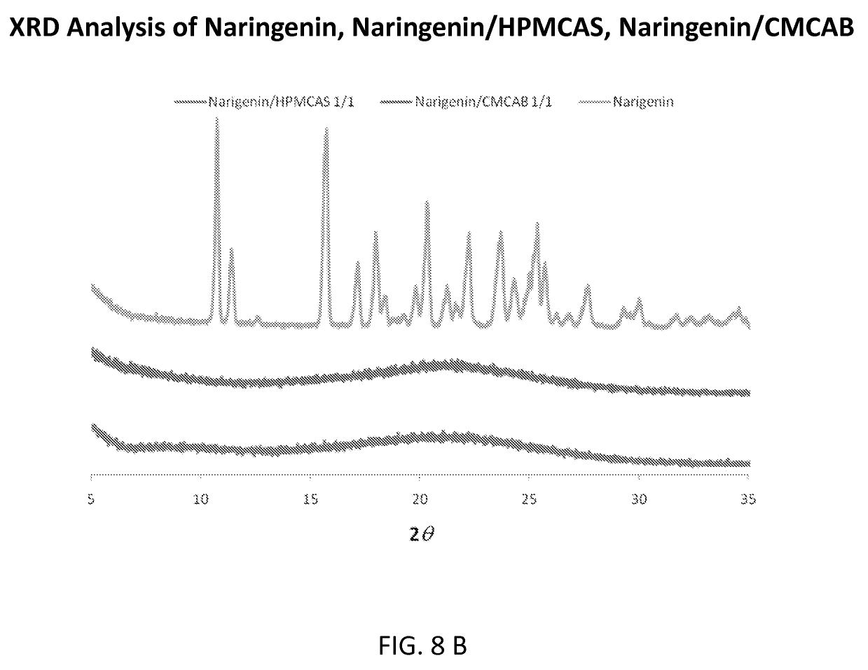

FIG. 8 shows various blends of ellagic acid with HPMCAS or PVP at various concentrations of ellagic acid in the blend. As shown in FIG. 8, ellagic acid blends with HPMCAS or PVP are amorphous up to about 50% concentration of ellagic acid in the blend. In preparing the compositions prior to using spray dry techniques, it may be desired to use a solvent other than acetone/ethanol (1/4), or it may be desired to add the flavonoid after dissolving the polymer in the solvent. For example, in preparing compositions of ellagic acid, it may be preferred to dissolve the polymer, such as CMCAB, in the solvent first then add the ellagic acid which will dissolve in the mixture even though ellagic acid is insoluble in acetone:ethanol 1:4 alone. Embodiments of the invention thus include compositions comprising ellagic acid in CMCAB, wherein the ellagic acid is added to CMCAB which is first dissolved in a solvent. As shown in FIG. 8A, XRD results indicate that naringenin was totally amorphous in the solid dispersion samples with concentrations of up to about 50% in HPMCAS or CMCAB.

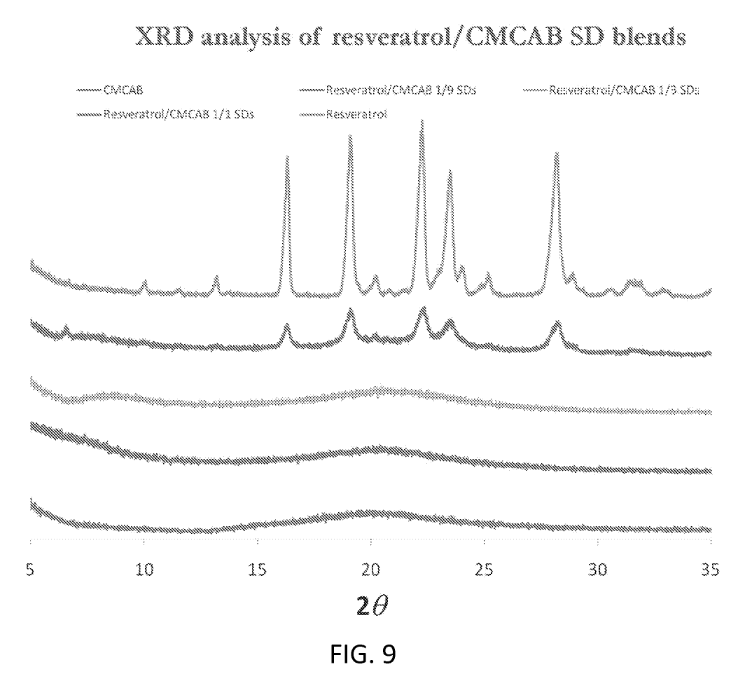

Yet another example is shown in FIG. 9, which illustrates that resveratrol is amorphous up to about 25% concentration in CMCAB spray dried blends, and that similar results are obtained with HPMCAS, HPMCP, or PVP. Some residual crystallinity in 25% blends in CAPh is observed.

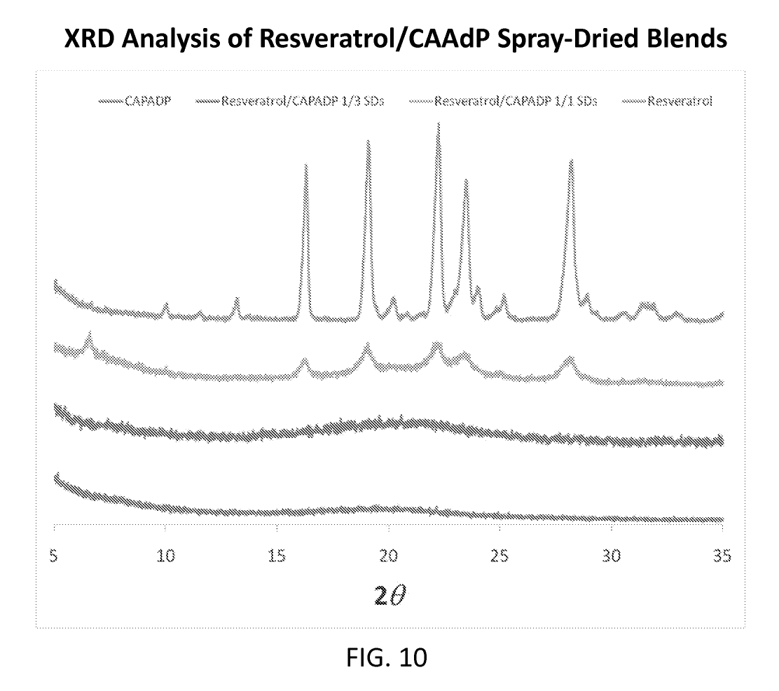

FIG. 10 shows that resveratrol is amorphous in CAAdP spray dry blends up to about 25 wt % and partly amorphous at about 50 wt %.

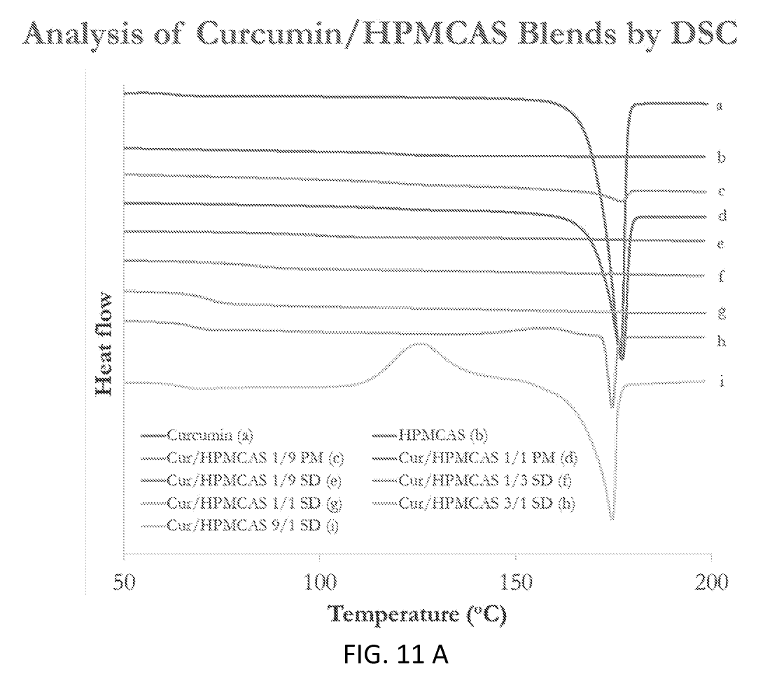

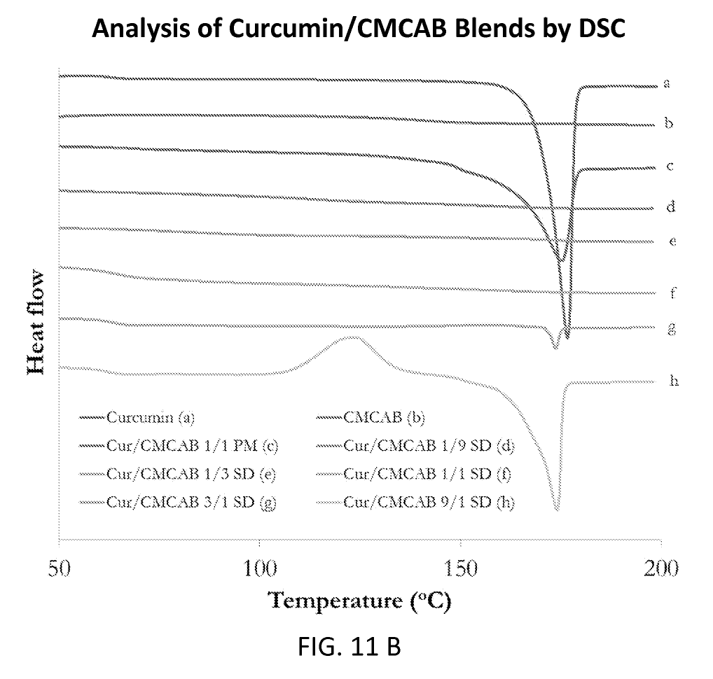

FIG. 11 shows analysis of various curcumin/HPMCAS blends by Differential Scanning calorimetry (DSC). Partially or completely amorphous solids exhibit a glass transition temperature and can exhibit a melt transition. Knowing glass transition temperatures is important for pharmaceutical stability storage and can be precisely determined with DSC. Changes in enthalpy of fusion detected by DSC, in comparison to a fully crystalline sample, can be used to determine the degree of crystallinity in an amorphous sample. As shown in FIG. 11, curcumin is amorphous at concentrations of up to about 50% in a spray dried blend with HPMCAS (e), (f), and (g), whereas concentrations of up to about 75% and 90% curcumin in a spray dried blend with HPMCAS are partly amorphous (h), (i). Compared with physical mixtures (c) and (d) which are partly amorphous, the molecular dispersion of curcumin with HPMCAS at the same concentrations, respectively (e) and (g), are completely amorphous.

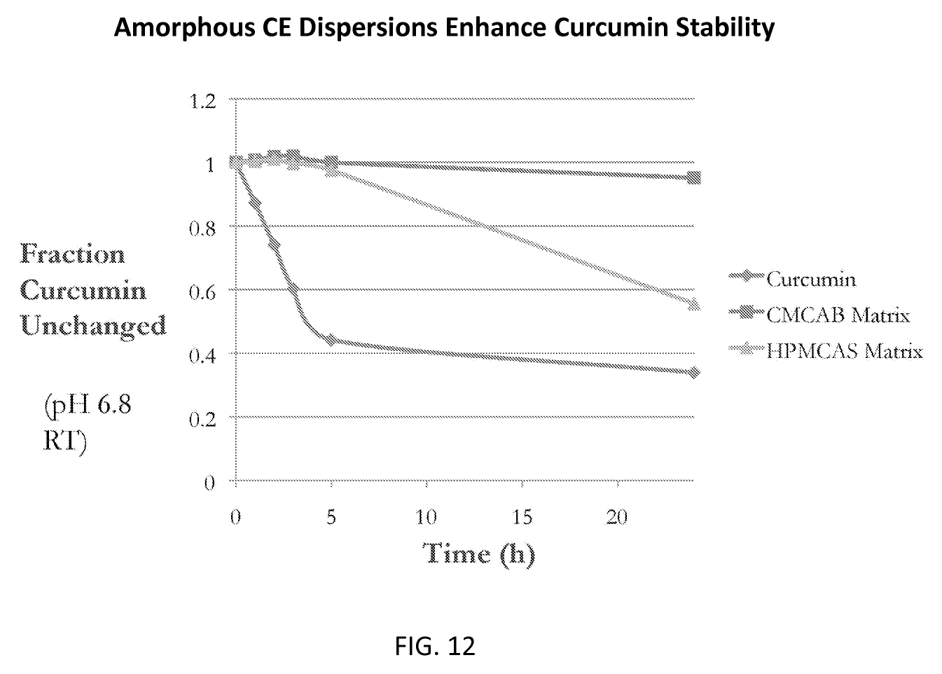

FIG. 12 provides a comparison of the stability of curcumin alone and in a CMCAB or HPMCAS matrix. As shown, curcumin is unstable at small intestine pH of about 6.8, whereas HPMCAS and especially CMCAB and CAAdP protect curcumin against solution degradation.

Formulation options for the flavonoid/cellulose derivative solid dispersions can be prepared by any known methods, including but not limited to: direct compression, thermal extrusion, co-precipitation, cast film and grind; and lyophilized or spray-dried from solution. Incorporating adjuvants, nanoparticles, liposomes, micelles, metabolic and efflux inhibitors, and phospholipid complexes may provide additional benefits to formulations of the invention to provide longer circulation, better permeability, and resistance to metabolic processes. Additionally, the use of piperine as metabolic inhibitor is another interesting approach, which may improve curcumin oral bioavailability up to 20-fold in humans due to its inhibition of glucuronidation. (Shoba 1998). Most recently Wu et al. reported formulation of curcumin in a self-microemulsifying drug delivery system (SMEDDS) composed of 20% ethanol, 60% Cremophor RH40 (R), and 20% isopropyl myristate, from which the concentration of curcumin reached 50 mg/mL and the relative oral bioavailability of SMEDDS compared with curcumin suspension was 1213%. (Phaechamud 2010).

In addition to pharmaceutical applications, there are potential nutraceutical uses for compositions according to embodiments of the invention, for example the compositions may be formulated into supplements sold in health food stores and pharmacies.

### Example I

Particular features of the present invention will now be described in the context of preparing and providing compositions comprising compounds, which are generally characterized by having high permeability but low solubility in non-amorphous form, in a solid dispersion within a polymer matrix where both are amorphous or partly amorphous. Such compositions, as explained in more detail below, are characterized by having one or more of improved solubility, stability, or bioavailability as compared with the non-amorphous form of the compound without the benefit of the polymer matrix. In particular, while a preferred compound curcumin is discussed below in more detail, this disclosure should not be interpreted as being limited to demonstrating only the feasibility of curcumin. Indeed, compounds with similar characteristics and properties to curcumin and any Class II drug can be substituted for curcumin to prepare similar inventive compositions. This disclosure is merely intended to provide direction to the ordinary skilled artisan to accomplish these goals.

Solubility enhancement of curcumin using spray dried solid dispersion with three cellulose derivatives (HPMCAS, CMCAB, and cellulose acetate adipate propionate (CAAdP), a new cellulose ester specifically designed as an effective polymer for molecular dispersions (Kar, N.; Liu, H.; Edgar, K. J. Synthesis of Cellulose Adipate Derivatives, Biomacromolecules 2011, 12, (4), 1106-1115)) as amorphous matrix polymers was investigated.

Curcumin, 1,7-bis(4-hydroxy-3-methoxyphenyl)-1,6-heptadiene-3,5-dione, is a hydrophobic polyphenol derived from the rhizome of turmeric (Curcuma longa). Curcumin exhibits keto-enoltautomerism as shown in Formula II:

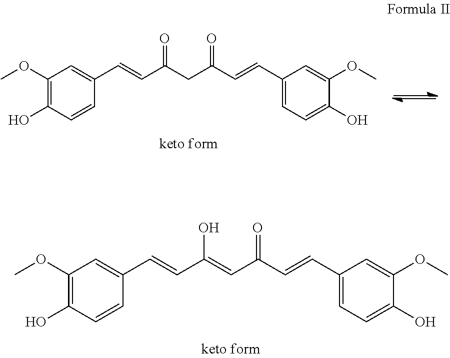

More particularly, the inventors evaluated the relative ability of these polymers to stabilize amorphous curcumin in the solid phase, to promote dissolution, and to stabilize curcumin against crystallization in the solution phase, all versus the curcumin/polymer ratio in the blend. The results were compared with those of pure curcumin and of Cur/PVP solid dispersions. Also observed was the unexpected ability of these cellulose derivatives to stabilize curcumin against chemical decomposition in solution.

In summary, curcumin (Cur) and the cellulose esters CMCAB, CAAdP and HPMCAS were readily blended by spray-drying, affording solid dispersions which were amorphous even at very high Cur levels. Release from CMCAB and CAAdP dispersions was quite slow and incomplete, probably due to the low wettability and low water solubility of these polysaccharide derivatives (these release rates can be enhanced using formulation techniques known to practitioners of the art). In contrast, release from HPMCAS matrices was much faster; HPMCAS systems are comparable to PVP systems for solubilization and release of Cur, while showing better protection of Cur against stomach contents. Remarkably, HPMCAS, CAAdP, CMCAB and PVP amorphous dispersions all not only inhibited Cur crystallization in solution but also protected Cur against the chemical degradation to which it is quite prone, with CMCAB and CAAdP clearly affording the best chemical stabilization. Systems based on the polysaccharide esters HPMCAS, CMCAB and CAAdP are promising for development of enhanced-bioavailability, curcumin-based therapeutic and dietary supplement formulations.

Chemicals.

Curcumin (98+%), PVP(K29-32, MW 58,000) and potassium bromide (99+%, for spectroscopy, IR grade) were supplied by Acros Organics (Geel, Belgium). CMCAB (641-0.2) was graciously provided by Eastman Chemical. HPMCAS (AS-LG) was generously supplied by Shin-Etsu Chemical Co., Ltd. (Tokyo, Japan). CAAdP was synthesized according to a previously reported method. Kar, N.; Liu, H.; Edgar, K. J., Synthesis of Cellulose Adipate Derivatives, Biomacromolecules 2011,12, (4), 1106-1115. Acetone (HPLC grade, 0.2 micron filtered), reagent alcohol (ethanol), potassium phosphate monobasic, and sodium hydroxide were supplied by Fisher Scientific (Fair Lawn, N.J.). Buffer solutions (pH 6.8, 7.4 and 1.2) were prepared according to the standard method in USP30-NF25.

Preparation of Spray-Dried Solid Dispersions.

The mixture of curcumin/matrix (10.0 g) in different weight ratios (1:9, 1:3, 1:1, 3:1 and 9:1) was dissolved in acetone/ethanol (1/4) to make a 20 g/L solution. Solid dispersions were prepared using a Buchi mini-spray dryer B-290 equipped with nitrogen purge for use with organic solvents. The operating parameters were: inlet temperature, 90° C.; outlet temperature, 57-60° C.; feed rate, 9 mL/min; nitrogen flow 350 L/h. The weight yield of the lab scale spray-drying process was ˜50-60%.

Physical mixtures were prepared to compare to the powders obtained by spray drying. The physical mixtures of curcumin and HPMCAS or CMCAB were made by grinding appropriate weights of the solids together in a mortar and pestle.

Characterization of the Spray-Dried Curcumin/Matrix Solid Dispersions.

Curcumin/polymer solid dispersions were characterized by comparing infrared (IR) spectra, nuclear magnetic resonance (NMR) spectra, differential scanning calorimetry (DSC) and X-ray powder diffraction (XRPD) patterns obtained for curcumin, polymer, physical mixture of curcumin/polymer, and spray-dried curcumin/polymer solid dispersions. Other techniques for analyzing crystallinity of the compositions may be used, including polarizing light microscopy.

IR Spectroscopy.

IR spectra were recorded in a frequency range between 4000 and 400 cm⁻¹, using a resolution of 4 cm⁻¹ and 40 accumulations, on a Nicolet 8700 FT-IR Spectrometer. FTIR pellets comprised 1 mg of the polymer matrix mixture and 100 mg of potassium bromide.

NMR Spectroscopy.

The NMR spectra were recorded on an Inova 400M instrument in acetone-d₆ with tetramethylsilane as internal reference.

XRPD Analysis.

XRPD measurements used a Bruker D8 Discovery X-Ray Diffractometer. The measurements were performed at a voltage of 40 kV and 25 mA. The scanned angle was set as 5<2θ<40° and the scan rate was 2°/min.

DSC Measurement.

Heating curves of curcumin and solid dispersion were obtained using a modulated differential scanning calorimeter (Model Q2000, TA Instruments, New Castle, Del.) equipped with a refrigerated cooling accessory. A 2-5 mg sample was packed in a non-hermetically crimped aluminum pan, and heated under dry nitrogen purge. Samples were heated from 25° C. to 100-120° C. at a heating rate of 10° C./min then quickly cooled to 25° C. at a cooling rate of 100° C./min to eliminate moisture and relieve stress, then heated from 25° C. to 200° C. at a heating rate of 10° C./min; transitions are reported from this second heating scan. DSC heating curves were analyzed using Universal Analysis 2000 software (TA Instruments).

Film Preparation.

The polymer matrix or curcumin solid dispersion solutions in acetone or ethanol in the concentration of 10 mg/mL were spin-coated onto silicon wafers at room temperature using a WS-400-6NPP/LITE spin coater (Laurell Technologies Corporation). The spinning velocity was 4,000 rpm and the spinning time was 60 s.

Contact Angle Measurements.

Contact angle measurements were carried out on the spin-coated film prepared as described above. The apparatus (FTA 200 dynamic contact angle analyzer, First Ten Angstroms, USA) was equipped with a magnifying video camera that automatically captured images of a water droplet during 8.5 s following initiation of the measurement.

UV-Vis Spectroscopy.

All UV-Vis spectra were recorded on a Thermo Scientific Evolution 300 UV-Visible Spectrometer.

Measurement of Solubility of the Matrix Polymers.

Polymer (0.5 g; CMCAB, HPMCAS or PVP) was dispersed in 10 mL of pH 6.8 buffer solution. The suspension was mixed by a vortex mixer for 1 min, followed by ultrasonicating for 15 min, then shaking for 24 h at room temperature by a Burrell wrist action shaker (Model 75). The suspension/solution was centrifuged at 14,000 g for 10 min to remove insoluble material. An aliquot (1 mL) of the top, clear solution was withdrawn and the solvent was evaporated in an oven at 80° C. for 5 h. The dissolved polymer weight could be calculated by subtracting the weight of salt in buffer solution (7.7 mg/mL calculated from the weight of potassium phosphate monobasic and sodium hydroxide in 1 L pH 6.8 buffer) from the weight of residue. The dissolved polymer concentration (w/v) could be then calculated by dividing the dissolved polymer weight by the volume of solution withdrawn.

Curcumin Calibration Curves in pH 6.8 and 1.2 Buffer.

A curcumin standard curve in ethanol was generated for the calculation of curcumin concentration from UV-Vis absorption. The effect of small amount (up to 5 v %) of water or aqueous buffer (pH 6.8) in ethanol was studied. The calibration curves in aqueous buffer (pH 6.8 or 1.2) were generated by dilution of a curcumin solution in ethanol (1.0 mg/mL, 10-200 μl) with pH 6.8 or 1.2 buffer solution to 10 mL. In order to study the effect of polymer matrix on the curcumin standard curve, the curcumin ethanol stock solution (1 mg/mL) was also diluted by the solution of PVP or HPMCAS (0.63 mg/mL) in pH 6.8 buffer or PVP (0.63 mg/mL) in pH 1.2 buffer. Due to the low solubility of CMCAB in pH 6.8 and 1.2 buffer and HPMCAS in pH 1.2 buffer, the standard curves of curcumin in the presence of CMCAB and HPMCAS in pH 1.2 buffer were not measured.

Dissolution Testing.

Curcumin solid dispersion (curcumin content was fixed at 50 mg) was dispersed in 10 mL of pH 6.8 phosphate buffer solution in an amber flask with magnetic stirring for 24 h. Then the suspension was centrifuged at 14,000 g for 10 min to remove any insoluble material. The curcumin concentration in the supernatant was determined by UV-Vis spectrometry using the calibration curve in ethanol generated as described above.

Enhancement of Curcumin Stability.

The stability enhancement of curcumin by polymers in solution was studied by UV-Vis spectrometry. Curcumin or curcumin/CMCAB (1/9) solid dispersion was dissolved in ethanol with curcumin concentration fixed at 1 mg/mL. The above ethanol solution (125 μl) was diluted to 10 mL with pH 6.8 or 7.4 buffer solution. Cur/HPMCAS and Cur/PVP (1/9, 1.25 mg) solid dispersions were dissolved directly (no EtOH) in 10 mL of pH 6.8 buffer solution. The UV-Vis absorption of the diluted solution was measured at certain time intervals from 0.5 to 24 h.

Aliquots of 50 μL, of 1 mg/mL curcumin or Cur/CMCAB 1/9 solid dispersion ([Cur]=1 mg/mL)(dissolved in ethanol) were added to 950 μl 0.2 M solutions of pH 7.4 buffer. Samples were incubated at room temperature for indicated times. After incubation, the reaction mixtures were diluted by 1 mL of ethanol and the UV-Vis absorption of the diluted solution was measured; then the diluted solution was filtered through a 1 μm PVDF membrane filter, and the filtrate was analyzed by HP LC. HPLC was performed with an Agilent 1200 series liquid chromatograph equipped with a 1200 quaternary pump, a variable wavelength UV/Vis detector and an Eclipse XDB-C18 column (150×4.6 mm, 5 μm particle size) using the mixture of 40% THF, 60% water and 1% acetic acid (pH 3.0) as mobile phase. Wang, Y.-J.; Pan, M.-H.; Cheng, A.-L.; Lin, L.-I.; Ho, Y.-S.; Hsieh, C.-Y.; Lin, J.-K., Stability of curcumin in buffer solutions and characterization of its degradation products, Journal of Pharmaceutical and Biomedical Analysis 1997,15, (12), 1867-1876.

Curcumin Release Profile.

Release of curcumin from dispersions was measured as follows. Curcumin samples (pure curcumin, physical mixture or solid dispersion) were dispersed in 100 mL pH 6.8 buffer solution in an amber glass flask with curcumin concentration of 0.07 mg/mL. The solution was stirred with a stir bar at 25° C. Aliquots (1.5 mL) were withdrawn at appropriate time intervals and replaced with 1.5 mL of fresh dissolution medium after each sampling to maintain constant volume. The UV-Vis absorption of the aliquots was recorded before and after centrifugation at 4550, 14000 or 70000 g for 10 min. To confirm removal of nanoparticles from the aqueous curcumin solution, the UV absorption of solutions from Cur/PVP 1/9 and Cur/HPMCAS 1/9 solid dispersions was measured again after filtering through a 0.2 μm pore size membrane filter. The drug release profiles of curcumin, Cur/PVP 1/9, Cur/HPMCAS 1/9 and Cur/CMCAB 1/9 solid dispersions in pH 1.2 buffer were measured using the same method and the aliquots were centrifuged before UV-Vis measurement.

Results. The polysaccharides were chosen, and in the case of CAAdP, designed, to be effective at stabilizing amorphous curcumin and releasing it at the pH of the small intestine. Thus CMCAB, HPMCAS and CAAdP are fundamentally hydrophobic polymers (for miscibility with hydrophobic actives such as curcumin), composed of low toxicity components like cellulose, acetic acid, and adipic or succinic acid, with a pendent carboxyl group to provide not only pH-triggered swelling and active release, but also effective specific interactions with hydrogen-bonding groups on the active molecule, to promote molecular dispersion. It is postulated that, in addition to the characteristics elucidated above that allow amorphous matrix polymers to provide their key roles of stabilizing the active molecule in the solid phase, and releasing said molecule at the desired rate in the GI tract, the ideal candidate polymer will also have at least a small amount of solubility in pH 6.8 media that will permit it to help stabilize dissolved active after release and prior to permeation through the GI epithelium. The inventors evaluated the ability of these polysaccharides to perform these roles with respect to curcumin, and compared them to the popular amorphous dispersion polymer PVP.

Characterization of Spray Dried Amorphous Solid Dispersions.

Initially the inventors evaluated the relative abilities of the three polymers to stabilize curcumin against crystallization in the solid phase, by preparing spray-dried dispersions of various concentrations of curcumin in these polymers and comparing with physical blends of the same composition. The blends were characterized by XRD, FT-IR, DSC and NMR spectroscopy.

XRPD was used to investigate the morphology of the curcumin/polymer matrices. As can be seen from FIGS. 7A-B, the XRPD patterns of all curcumin/CMCAB physical mixtures are similar to that of crystalline curcumin, with intensity proportional to percent curcumin; this indicated the continuing presence of crystalline curcumin and the ability to detect it down to as little as 10% by weight. In contrast, XRPD patterns of all curcumin/CMCAB spray dried blends were smooth without any crystalline peaks, indicating that curcumin is completely amorphous in CMCAB molecular blends even up to 90% curcumin. Similarly, all curcumin/HPMCAS spray dried dispersions were completely amorphous, but crystalline peaks were observed in all the physical mixtures.

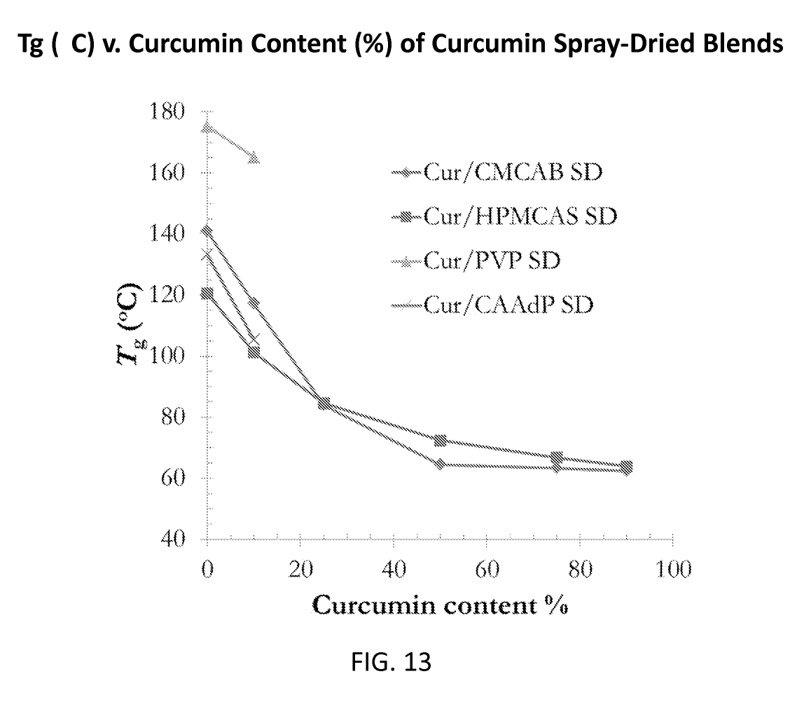

DSC is also a useful tool for investigating the morphology of active/polymer blends, and can give information not only about drug morphology in the blend, but also polymer morphology. In the case of polysaccharides containing both pendent carboxyl and hydroxyl groups (like CMCAB, HPMCAS, and CAAdP) DSC transitions are observed above 200° C. that can be ascribed to crosslinking esterification reactions between those pendent groups. Therefore it is important to carry out DSC analyses of mixtures containing these polymers at temperatures not greater than about 200° C. DSC heating curves of curcumin solid dispersions as well as those of pure curcumin and the individual matrix polymers are shown in FIGS. 11A-B, and the T_{g} values of curcumin solid dispersions are plotted versus curcumin content in FIG. 13.

The curcumin used in these studies displayed a melting transition at 177° C. The polymer matrices showed T_{g} of 141° C. for CMCAB, 133° C. for CAAdP, 121° C. for HPMCAS and 175° C. for PVP. By DSC the Cur/matrix physical mixtures had T_{g} values similar to those of the pure polymer, and melting transitions similar to that of pure curcumin.

In contrast (FIGS. 11A, 11B), the T_{g} of curcumin spray-dried dispersions decreased compared to that of the corresponding polymer matrix and decreased with increasing curcumin content, which is typical behavior of a small molecule plasticizer in a polymer matrix. At curcumin content ≦50%, no curcumin melting peak was observed, indicating that curcumin was amorphous and protected against crystallization even at high temperature. Interestingly curcumin melting endotherms were observed for Cur/CMCAB and Cur/HPMCAS solid dispersions with curcumin content ≧75%, and in all four cases (Cur/CMCAB or Cur/HPMCAS, 3/1 and 9/1) exothermic curcumin crystallization peaks were observed as well. Curcumin melting transitions at curcumin content higher than 75% shifted from 176 to around 170° C., which may indicate less perfect crystals than for pure curcumin The contrast between XRPD and DSC results may indicate the potential for high temperature curcumin crystallization at ≧75% curcumin, rather than the presence of detectable crystalline curcumin in these blends at ambient temperatures. Similar DSC changes were observed in the Cur/PLGA (poly(lactic/glycolic) acid) system. Shahani, K.; Panyam, J., Highly loaded, sustained-release microparticles of curcumin for chemoprevention, J Pharm Sci-US 2011, 100, (7), 2599-2609.

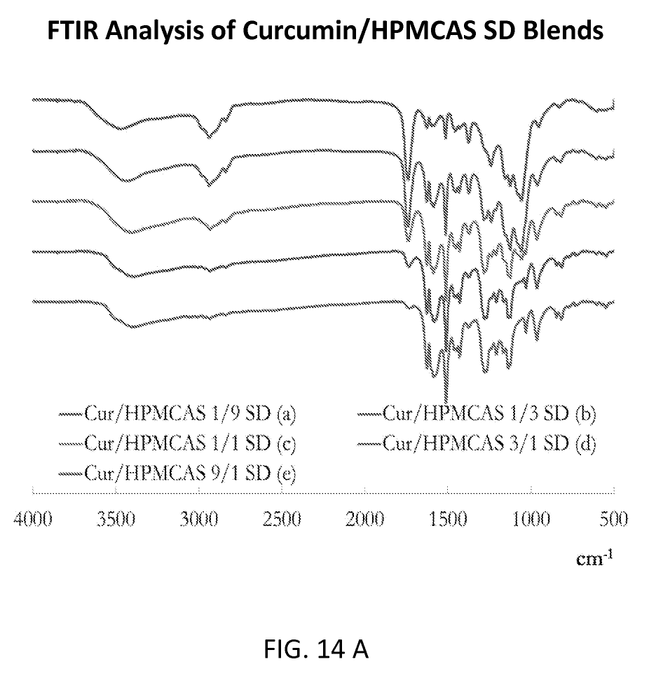

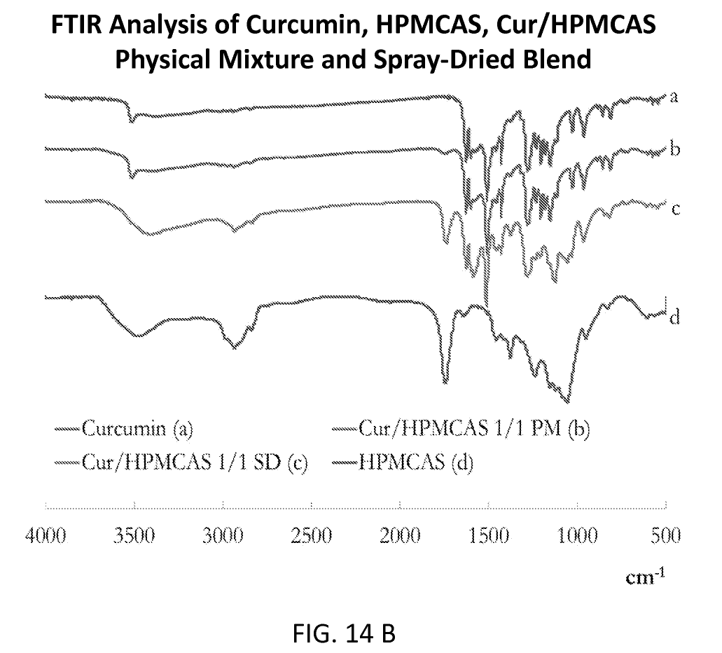

FTIR was used to explore curcumin-polymer interactions in the matrix. IR spectra of curcumin, HPMCAS, Cur/HPMCAS (1:1) physical mixtures and spray dried solid dispersions are shown in FIGS. 14A-B. The band at 3500 cm⁻¹ was attributed to curcumin —OH stretching. In the physical mixture this band shape does not change and the sharp peak at 3500 cm⁻¹ can be observed clearly. However, the spray dried dispersion shows broad peaks at 3600-3400 cm⁻¹, similar to the bands of pure HPMCAS. This significant broadening of peaks may be attributed to the intermolecular hydrogen bonding between curcumin and the matrix polymer, which may aid the disruption of the curcumin crystalline structure. HPMCAS alone shows a strong absorption at 1747 cm⁻¹ due to C═O stretch. The spray-dried HPMCAS/curcumin showed slight shifts in this peak to 1740 cm⁻¹, also indicative of the hydrogen bonding interaction between curcumin and matrix polymer.

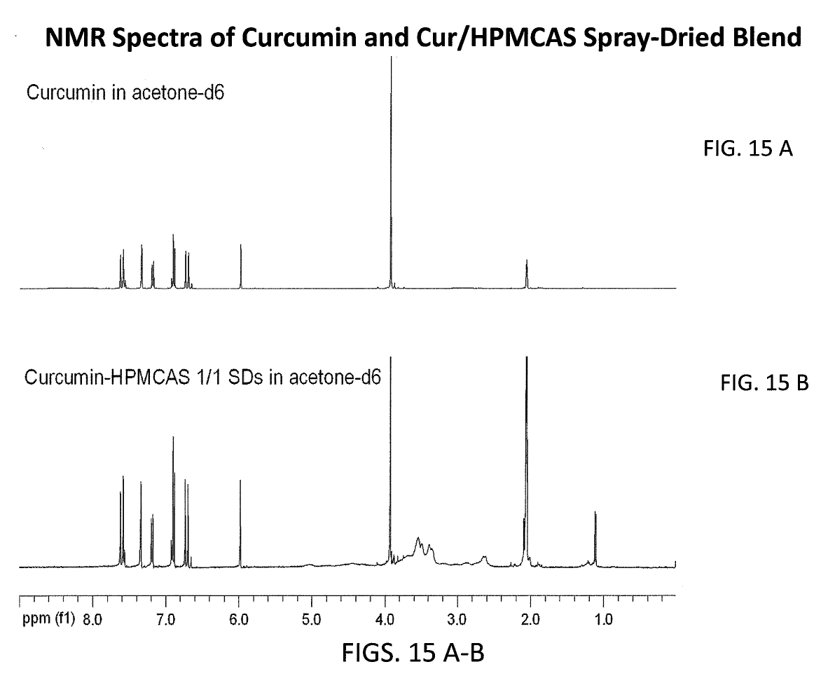

In order to confirm that there is no chemical reaction between curcumin and the matrix, the ¹H-NMR spectra of spray dried solid dispersions were recorded. The spectra are provided in FIGS. 15A-B. The spectra are simply additive of those of pure curcumin and matrix polymer, with no new peaks or significant shifts in resonances. These results support the conclusion that flavonoids, e.g., curcumin, is stable under the spray drying conditions, and not reactive with the matrix polymers.

Contact Angle of Water on the Solid Dispersion Film.

The inventors studied the contact angles of water with curcumin and curcumin molecular dispersions, as spin-coated films, with the idea that wettability might also influence dissolution rate from these dispersions. The contact angles of water on the pure component films are: curcumin 102°, CMCAB 69.9° (Lit 69.5)° (Amim, J.; Petri, D.; Maia, F.; Miranda, P., Solution behavior and surface properties of carboxymethylcellulose acetate butyrate, Cellulose 2009, 16, (5), 773-782), HPMCAS 58.9° and PVP 33.5°. As can be seen from Table 2, the water contact angles of the curcumin solid dispersions are always higher than that of the pure polymer but much lower than that of pure curcumin; the water contact angle increased with increasing curcumin content. Clearly the wettability of curcumin solid dispersions was influenced by both the concentration and nature of the polymer. Even the 90% curcumin solid dispersion displayed much lower water contact angle than that of pure curcumin. The water contact angles of PVP and Cur/PVP 1/9 solid dispersion decreased rapidly due to the water solubility of PVP, so it was difficult to study wettability using this method. However, the initial contact angle values of PVP (33.5°) and Cur/PVP 1/9 SD blend (48.9°) were lower than the corresponding HPMCAS or CMCAB samples, as expected given the water affinity of PVP.

TABLE 2
Contact angles of water with films of curcumin, polymer
matrices and Cur/polymer solid dispersions

| Substrate (Cur/polymer) | Cur/CMCAB | Cur/HPMCAS | Cur/PVP |
|---|---|---|---|
| 100/0 | 102 | 102 | 102 |
| 90/10 | 77.3 | 66.4 | — |
| 75/25 | 77.7 | 65.7 | — |
| 50/50 | 77.7 | 65.2 | — |
| 25/75 | 76.2 | 61.4 | — |
| 10/90 | 74.3 | 60.4 | 48.9 |
| 0/100 | 68.9 | 58.9 | 33.5 |

Cur Calibration Curve.

Creation of a calibration curve for poorly soluble species like Cur is tricky, since it is difficult to reach required concentrations in the most pertinent medium, aqueous buffer, in the absence of stabilizing polymer. It has been reported that the maximum absorption of curcumin at ˜420-430 nm may be assigned to the π-π* transitions of the enolic form in solution. Shen, L.; Ji, H.-F, Theoretical study on physicochemical properties of curcumin, Spectrochimica Acta Part A: Molecular and Biomolecular Spectroscopy 2007, 67, (3-4), 619-623.

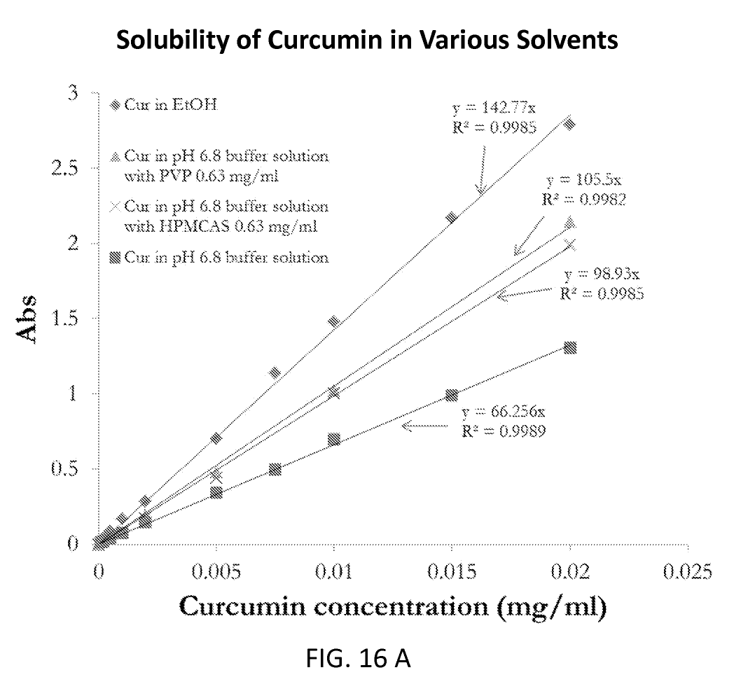

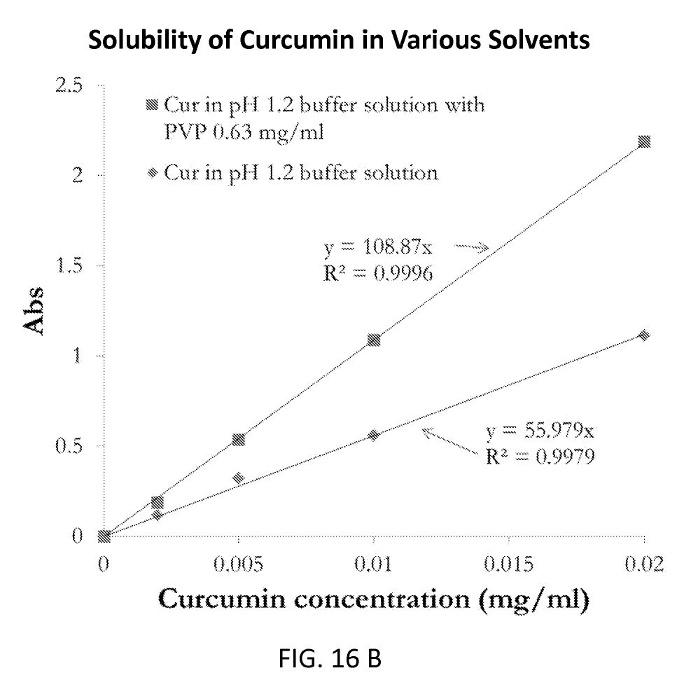

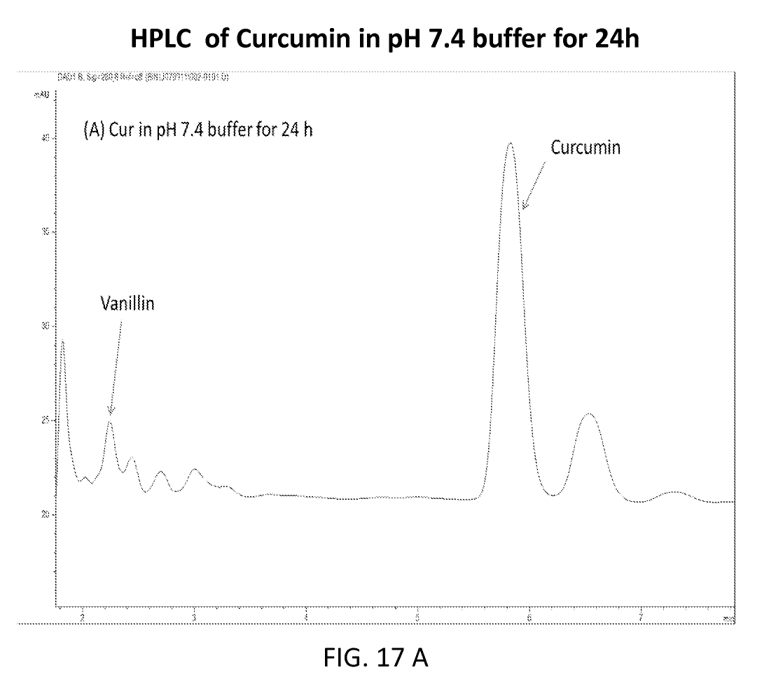

The stability of the curcumin excited state and the position of equilibrium between enolic and diketo form may be impacted by the nature of the solvent. From the standard curves of curcumin shown in FIGS. 16A-B, the extinction coefficient of curcumin is significantly changed from 142 L·g⁻¹·cm⁻¹ in ethanol to 66 L·g⁻¹·cm⁻¹ in pH 6.8 buffer solution. Such a large difference cannot be explained only by the difference in solvent polarity, since both ethanol and water are strong polar solvents. The most likely reason is fast crystallization and/or degradation of curcumin upon dilution of curcumin ethanol solution with aqueous buffer. The similar coefficients of curcumin in pH 6.8 and 1.2 buffer indicate that crystallization may be the dominant factor, since stability of curcumin in pH 1.2 buffer is much higher than that in pH 6.8 buffer (vide infra). Interestingly, the extinction coefficient in pH 6.8 buffer increased to 99 and 106 L·g⁻·cm⁻¹ in the presence of 0.63 g/L of HPMCAS and PVP, respectively, presumably due primarily to their stabilization of Cur against crystallization. Neither PVP nor HPMCAS show any absorption at 427 nm UV/Vis calibration curves of Cur in ethanol were used to avoid issues arising from crystallization and nanoparticles.

Suppression of Curcumin Degradation.

It has been reported that curcumin is rapidly degraded in aqueous solution, with accelerating rates as the pH reaches 6.8 and above. Wang, Y.-J.; Pan, M.-H.; Cheng, A.-L.; Lin, L.-I.; Ho, Y.-S.; Hsieh, C.-Y.; Lin, J.-K., Stability of curcumin in buffer solutions and characterization of its degradation products, Journal of Pharmaceutical and Biomedical Analysis 1997, 15, (12), 1867-1876 (“Wang 1997”); and Priyadarsini, K. I., Photophysics, photochemistry and photobiology of curcumin: Studies from organic solutions, bio-mimetics and living cells, J Photoch Photobio C 2009, 10, (2), 81-95 (“Priyadarsini 2009”).

The products of curcumin degradation include trans-6-(4-hydroxy-3-methoxyphenyl)-2,4-dioxo-5-hexenal, vanillin, ferulic acid, and feruloyl methane. Formula III below illustrates the process of curcumin degradation.

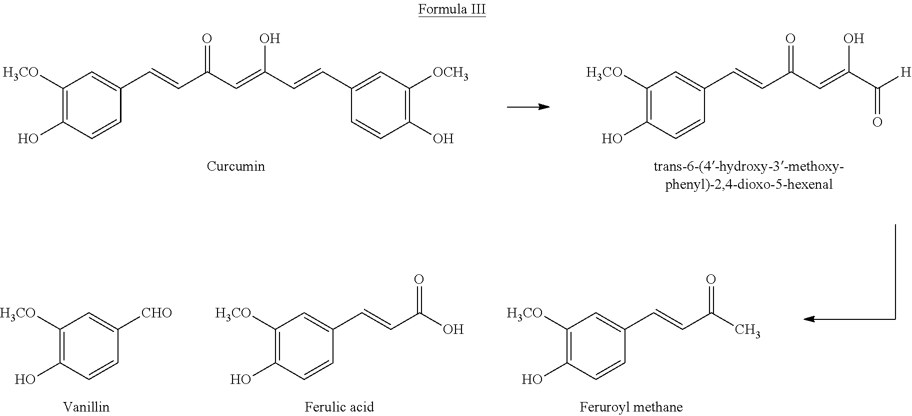 [SMILES: COc1cc(/C=C/C(=O)/C=C(O)/C=C/c2ccc(O)c(OC)c2)ccc1O ; COc1cc(/C=C/C(=O)/C=C(\O)C=O)ccc1O ; COc1cc(C=O)ccc1O ; COc1cc(/C=C/C(=O)O)ccc1O ; COc1cc(/C=C/C(C)=O)ccc1O]

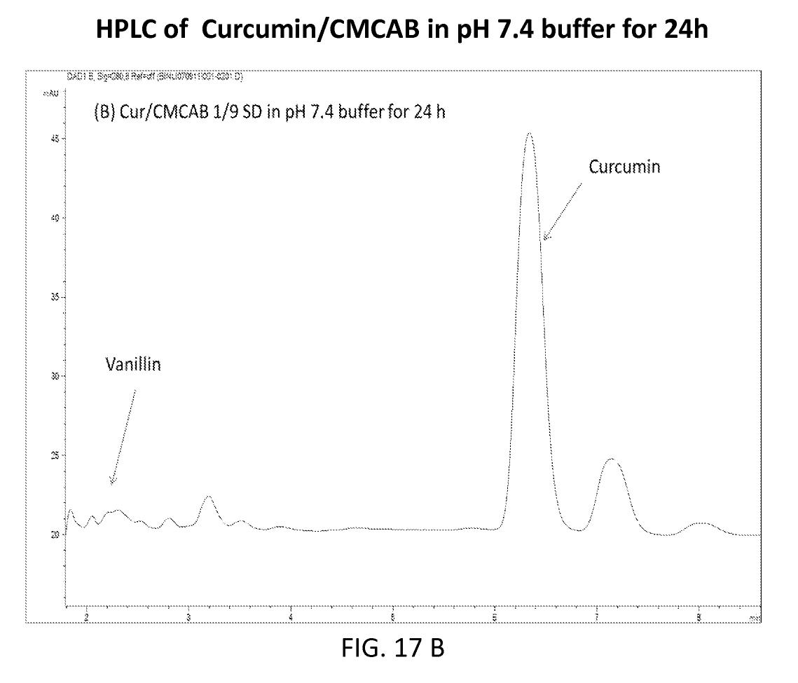

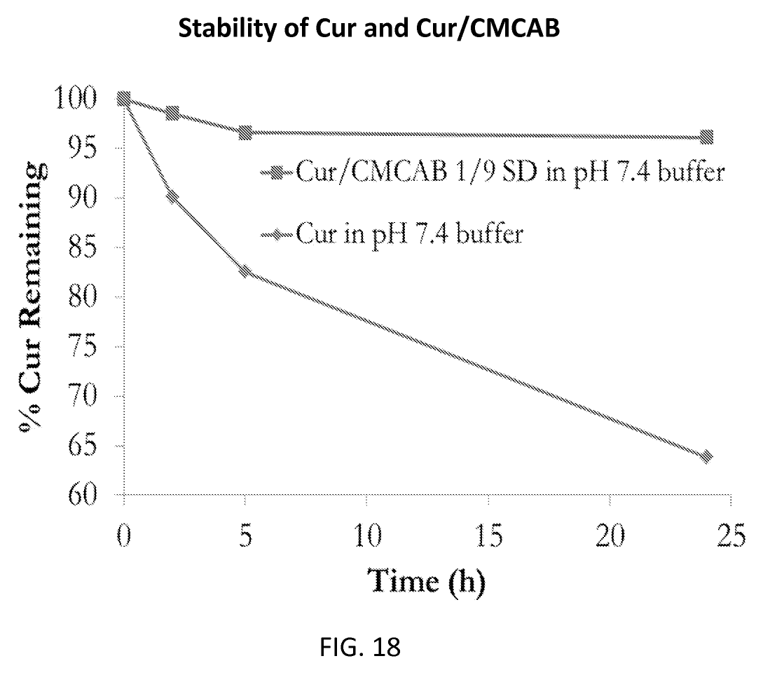

Wang and co-workers found that in pH 7.4 buffer, the half-life of curcumin is only 0.09 h, while at pH 6.8, degradation is slower but still rapid, with a T₁₁₂ of about 4 h. (Wang 1997). Such chemical degradation is likely the result of retro-aldol reactions. (Priyadarsini 2009). However, the inventors observed slower Cur degradation by HPLC and UV-Vis spectrometry, than that reported in the literature (Wang et al report (Wang 1997) ca. 90% curcumin degradation in pH 7.2 buffer at 37° C. within 30 min) As shown in FIGS. 17A-B, HPLC of curcumin in ethanol showed three peaks corresponding to curcumin (retention time (RT) 6.0 min), demethylcurcumin (RT 6.8 min), and bis-demethylcurcumin (RT 7.6 min). After incubation in aqueous buffer (pH 7.4 or 6.8) for 24 h, several new peaks appear with RT from 2 to 3.5 min, which correspond to curcumin degradation compounds including vanillin (RT 2.14 min) and ferulic acid (RT 2.03 min), while the peaks corresponding to curcumin are still strong. As shown in FIG. 18, calculation based on the integration of the curcumin peak at RT 6.0 min showed that only 36% of curcumin degraded after incubation in pH 7.4 buffer for 24 h at room temperature. This experiment differs from the literature report in that the inventors use ethanol to dilute the incubated aqueous curcumin solution just prior to measurement, to make sure that any crystallized curcumin is re-dissolved for detection. The results demonstrate that curcumin crystallizes in aqueous buffer, and that the crystallized curcumin degrades more slowly; it is important in Cur stability studies to carefully separate the effects of crystallization and degradation. As also shown in FIG. 18, in contrast to pure Cur, Cur/CMCAB 1/9 SD shows enhanced Cur chemical stability in solution, with only 4% curcumin degradation detected after 24 h in pH 7.4 buffer.

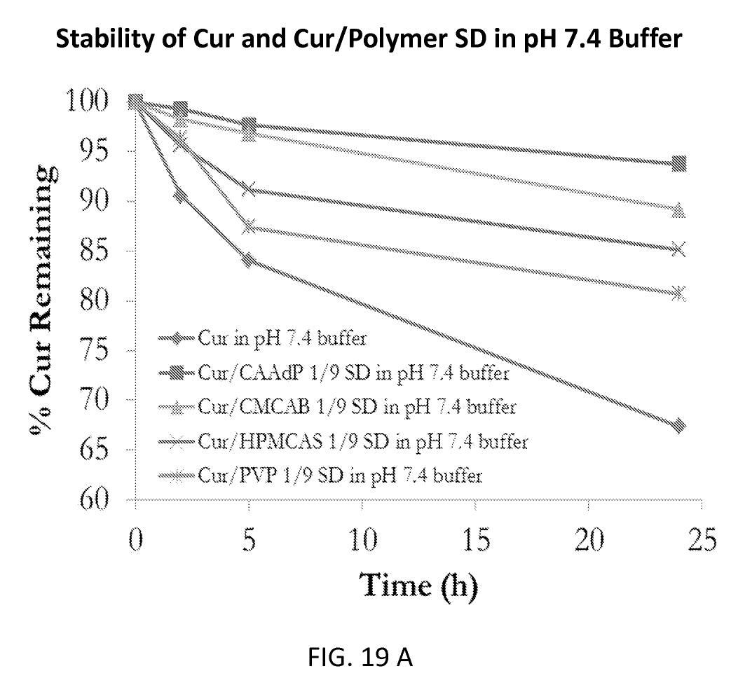

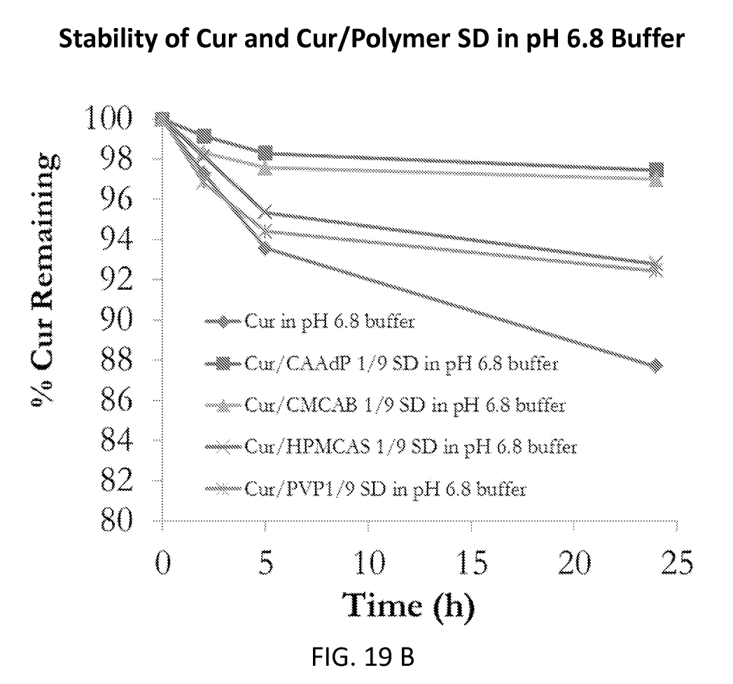

The impact of the polymers on curcumin degradation was further confirmed by UV-Vis spectroscopy of the mixture of curcumin in aqueous buffer (pH 7.4); individual samples for each time point were diluted with an equivalent volume of ethanol just prior to measurement. As shown in FIGS. 19A-B, after 24 h in pH 7.4 buffer, pure Cur has degraded by 33%, while Cur in a solid dispersion with CMCAB (1/9 respectively) has degraded only 11%. Similar stabilization is observed at pH 6.8. These results confirmed that CMCAB stabilizes curcumin and inhibits its degradation in aqueous buffer. Analysis of curcumin solid dispersions in the other polymer matrices under the same conditions indicated that ability to inhibit curcumin degradation follows the following sequence: CAAdP>CMCAB>HPMCAS>PVP. This chemical stabilization, like stabilization against crystallization, is presumably due to solution interaction between the polymer and curcumin.

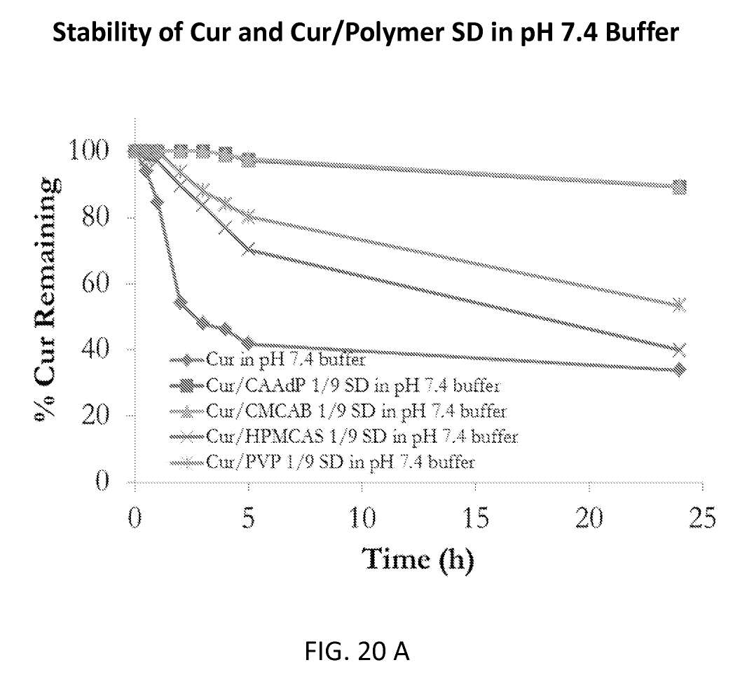

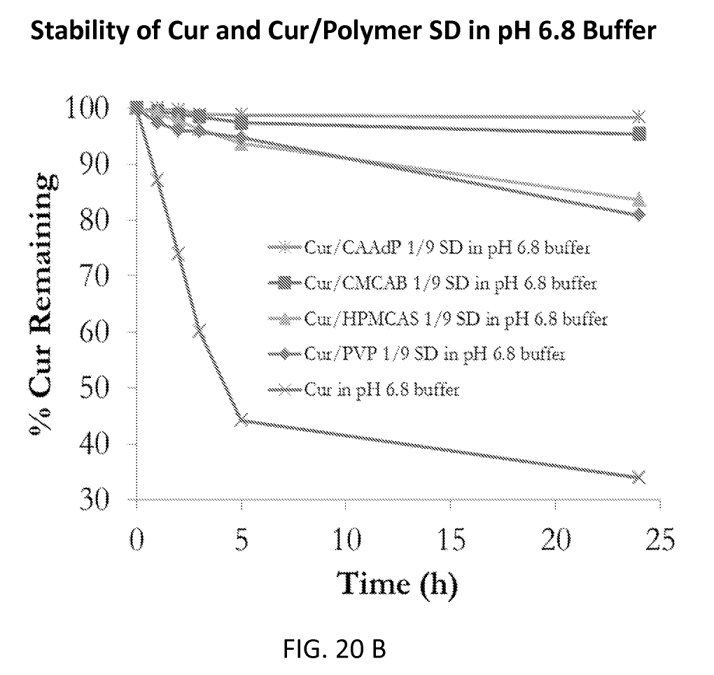

The contribution of precipitation to the removal of Cur from solution is illustrated in FIGS. 20A-B, which shows UV-Vis absorption of Cur solid dispersions in pH 7.4 and 6.8 buffer (without ethanol dilution) versus time. Dissolved Cur in both pH 6.8 (FIG. 20A) and 7.4 (FIG. 20B) buffers decreased rapidly and only around 40% (pH 7.4) and 44% (pH 6.8) of curcumin remained in solution after 5 h. However, as shown in FIGS. 19A-B, Cur degrades far less than this after 5 h (7% at pH 6.8, 16% at pH 7.4), indicating that precipitation is the dominant effect on removal of Cur from solution under these conditions, and further illustrating the remarkable importance of stabilization by polymers against crystallization. While 66% of pure curcumin in pH 6.8 buffer solution had degraded or crystallized within 24 h, spray-dried molecular dispersions of curcumin in PVP (19%) and HPMCAS (16%) degraded or crystallized to a much lesser extent over the same time period. The more hydrophobic CMCAB and CAAdP stabilized curcumin against chemical degradation and crystallization in pH 6.8 buffer solution almost completely, with 95-98% of curcumin remaining intact after 24 h. This combined stabilization against degradation and precipitation is unprecedented. In fact, dissolution of molecularly dispersed Cur in HPMCAS has been observed to accelerate photochemical degradation in comparison with that of crystalline Cur, and stabilize it only slightly in comparison with dissolved pure Cur. Onoue, S.; Takahashi, H.; Kawabata, Y.; Seto, Y.; Hatanaka, J.; Timmerman, B.; Yamada, S., Formulation Design and Photochemical Studies on Nanocrystal Solid Dispersion of Curcumin with Improved Oral Bioavailability, J. Pharm. Sci. 2010, 99, (4), 1871-1881.

Very recently, investigators have reported protection of the related flavonoid quercetin from degradation in alkaline solution by formulation as lecithin-based nanoparticles. Date, A. A.; Nagarsenker, M. S.; Patere, S.; Dhawan, V.; Gude, R. P.; Hassan, P. A.; Aswal, V.; Steiniger, F.; Thamm, J.; Fahr, A., Lecithin-Based Novel Cationic Nanocarriers (Leciplex) II: Improving Therapeutic Efficacy of Quercetin on Oral Administration (dagger), Molecular Pharmaceutics 2011, 8, (3), 716-26. Solution complexation of the polymer with the active molecule is essential to prevent recrystallization of the active. It is known that these polymers can dissolve in pH 6.8 buffer at μg/mL levels (even though the bulk of CMCAB for example does not dissolve at that pH). Alonzo, D. E.; Gao, Y.; Zhou, D.; Mo, H.; Zhang, G. G. Z.; Taylor, L. S., Dissolution and precipitation behavior of amorphous solid dispersions, J Pharm Sci-Us 2011, n/a-n/a; and Van Eerdenbrugh, B.; Taylor, L. S., Small Scale Screening To Determine the Ability of Different Polymers To Inhibit Drug Crystallization upon Rapid Solvent Evaporation, Molecular Pharmaceutics 2010, 7, (4), 1328-1337; and Rumondor, A.; Stanford, L.; Taylor, L., Effects of Polymer Type and Storage Relative Humidity on the Kinetics of Felodipine Crystallization from Amorphous Solid Dispersions, Pharmaceutical Research 2009, 26, (12), 2599-2606.

Such solution complexation may also be the source of the chemical stabilization. Polysaccharide complexation with smaller organic molecules is well-known and quite selective in the case of the amylose helix or of the cavity of cyclodextrins, but such complexation is not well-known for cellulose derivatives. The observed stabilization addresses a major previous drawback to the therapeutic use of curcumin.

Dissolution Testing and Solubility Enhancement.

The extremely low water solubility of curcumin and the expected supersaturation achievable from molecular dispersion in polysaccharide matrices make for complex equilibria and analytical problems. For example, generating a calibration curve for curcumin that covers supersaturated concentrations is of course problematic, in particular, it is difficult to generate such concentrations in the absence of polymer. The generation of nanoparticles is also problematic; these may be formed by recrystallization from the supersaturated solutions, for example. Such particles are difficult to remove by filtration; even 20 nm syringe filters may not catch the smallest nanoparticles, and filtration is also problematic since nonpolar solvates may adsorb onto filter media. Lindenberg, M.; Wiegand, C.; Dressman, J. B., Comparison of the adsorption of several drugs to typical filter materials, Dissolution Technol. 2005, 12, (Copyright © 2011 American Chemical Society (ACS). All Rights Reserved.), 22-25. Additionally, nanoparticles can absorb UV light and therefore may be erroneously counted as dissolved active. Bohren, C. F.; Huffman, D. R., Absorption and Scattering of Light by Small Particles, ed.; John Wiley and Sons: 1983; p 530.

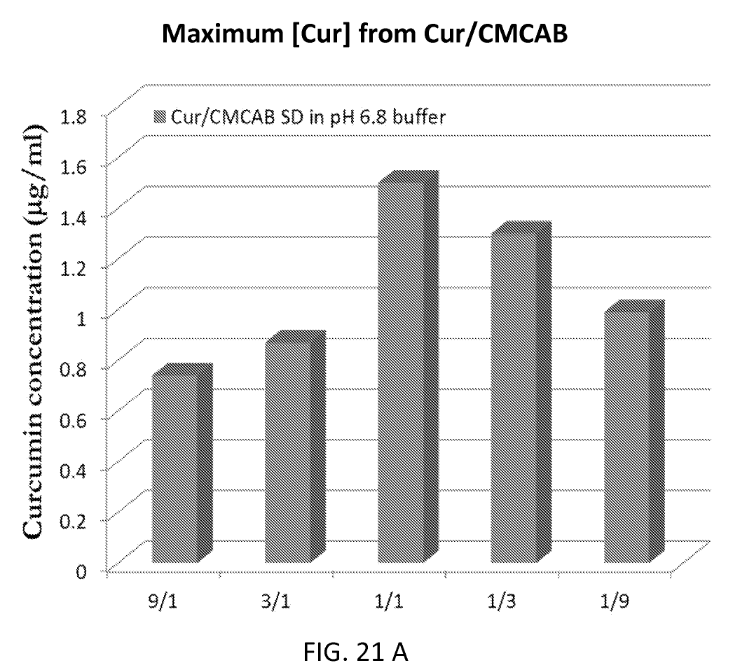

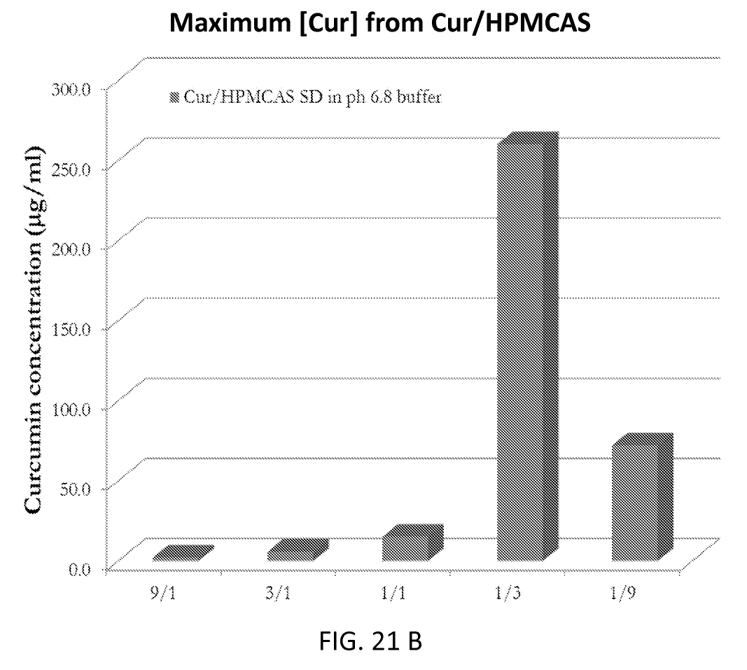

The inventors dealt with these issues by generating calibration curves with solutions of curcumin in ethanol. Control experiments were carried out to show that the extinction coefficient of curcumin was not substantially changed by the addition of small amounts of water (up to 5 volume %, because adding more may result in the possibility of partial Cur crystallization). Any nanoparticles present were removed by centrifugation of solutions prior to UV-Vis measurements. This protocol gave highly repeatable values with an error less than 5%. In the dissolution tests, curcumin solid dispersion was dispersed in pH 6.8 buffer solution with a fixed maximum curcumin concentration of 5 mg/mL. After shaking until equilibrium was reached (as determined by a measured plateau in [Cur]) then centrifuging, the curcumin concentration was measured by UV-Vis analysis of the supernatant, which is shown in FIGS. 21A-B. Clearly molecular dispersions with lower curcumin concentration lead to higher curcumin solubilization when the polymer amount in the SD samples is less than its maximum solubility, as is typical of amorphous dispersion formulations. Kaewnopparat, N.; Kaewnopparat, S.; Jangwang, A.; Maneenaun, D.; Chuchome, T.; Panichayupakaranant, P., Increased Solubility, Dissolution and Physicochemical Studies of Curcumin-Polyvinylpyrrolidone K-30 Solid Dispersions, World Academy of Science, Engineering and Technology 2009, 55, 229-234. It is also clear that solubility from HPMCAS dispersions is uniformly much higher than from equivalent CMCAB dispersions. One influence on the maximum Cur solubility attainable from these amorphous solid dispersions is the relative aqueous solubility of the four polymers.

TABLE 3
Solubility of polymers and maximum
Cur solubility from their ASDs

| | | maximum Cur solubility |
|---|---|---|
| | solubility in water | from amorphous |
| polymer | (mg/mL) | dispersion (μg/mL) |
| PVP | >600 | 2900 |
| HPMCAS | 23.4 | 260 |
| CMCAB | 1.6 | 1.5 |
| CAAdP | 1.5 | 1.4 |

The solubility of the matrix polymer will influence active solubility in at least two ways. The partial or total swelling and dissolution of the polymer matrix can serve as a drug release mechanism. In addition, the critical polymer function of stabilizing the active against crystallization from supersaturated solution, already mentioned, relies upon some polymer dissolution. The wettability of the polymer matrix is also dependent on polymer hydrophobic/hydrophilic balance, and is likely also to have an effect on active dissolution.

Drug Release Profiles.

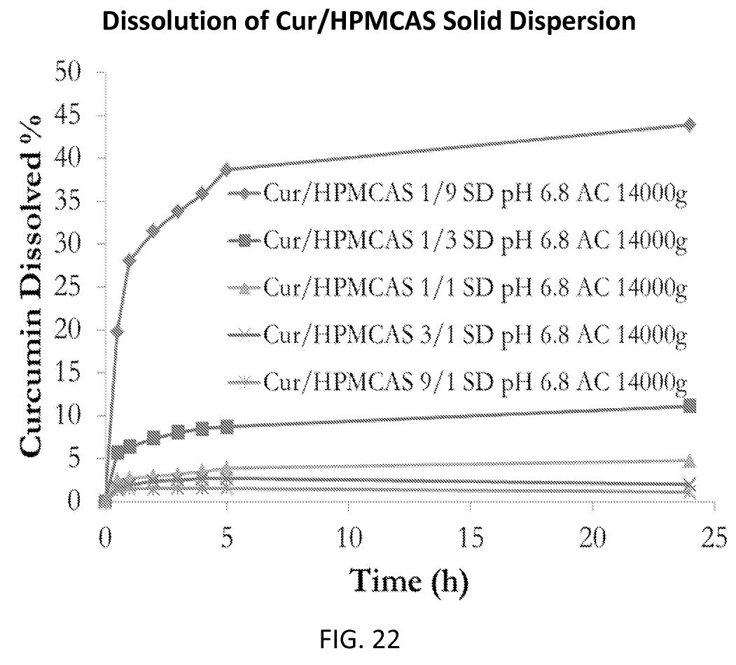

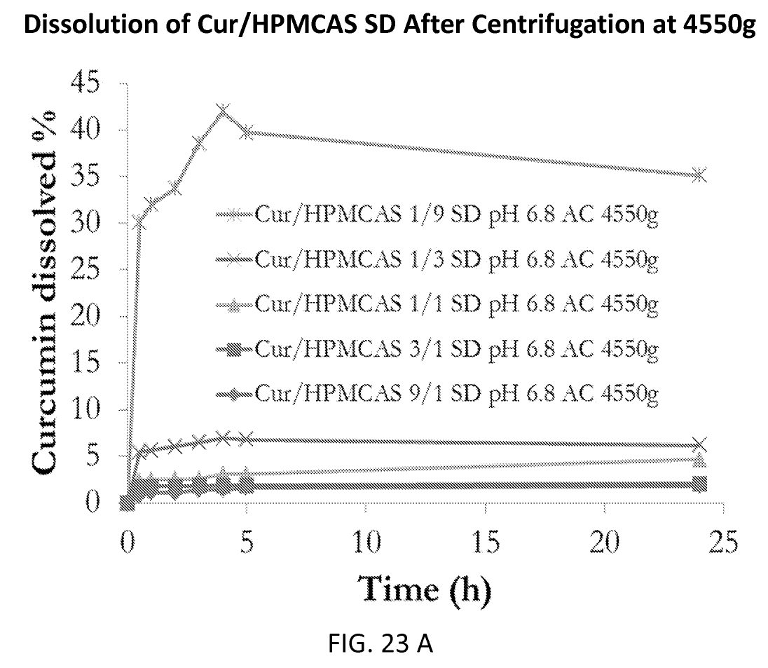

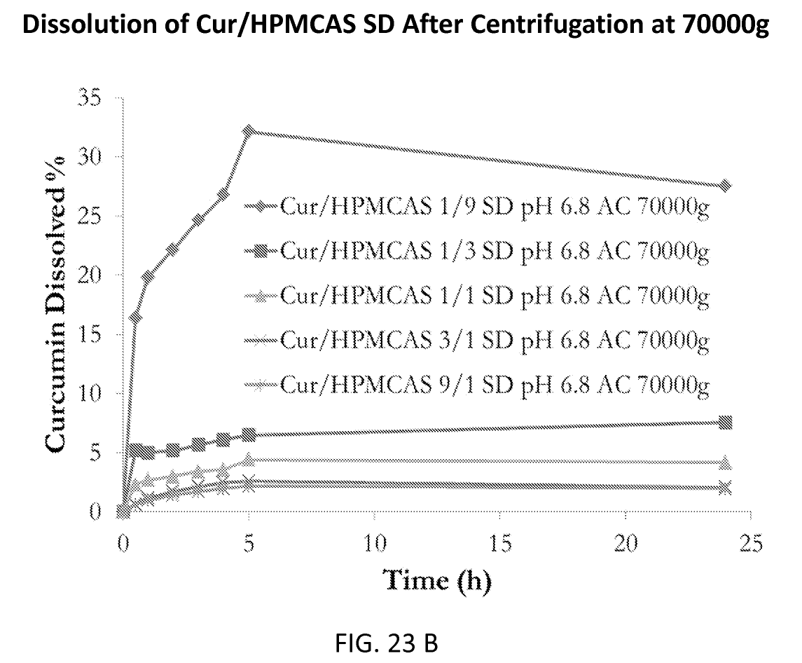

In measuring drug release profiles, the maximum Cur concentration was fixed at 0.07 mg/mL in pH 6.8 buffer solution. The UV-Vis absorption of the solution was measured and plotted vs. time. Centrifugation (14,000 g, 10 min) was employed to remove suspended solid from the dissolution experiment samples (the inventors opted not to use filtration because of possible Cur adsorption to filter media as noted above); aliquots of the clear centrifugates were analyzed by UV-Vis. Results are shown in FIG. 22. The fastest and most extensive Cur release was observed at the lowest Cur concentrations in the solid dispersions. Release was also faster and more complete with HPMCAS as compared with CMCAB. At 1:9 curcumin/HPMCAS the highest release (42%) was observed within 5 h, vs. only 10% release from a 1:1 dispersion after 24 h. Three different centrifugation speeds (4,500, 14,000 and 70,000 g, 10 min) were used to ensure removal of all of the nanoparticles. As shown in FIG. 23, results were quite comparable at the three speeds, with slightly lower apparent solubility observed as centrifugation speed increased. It was more convenient to use 14,000 g, so this standard centrifugation speed was used for other experiments.

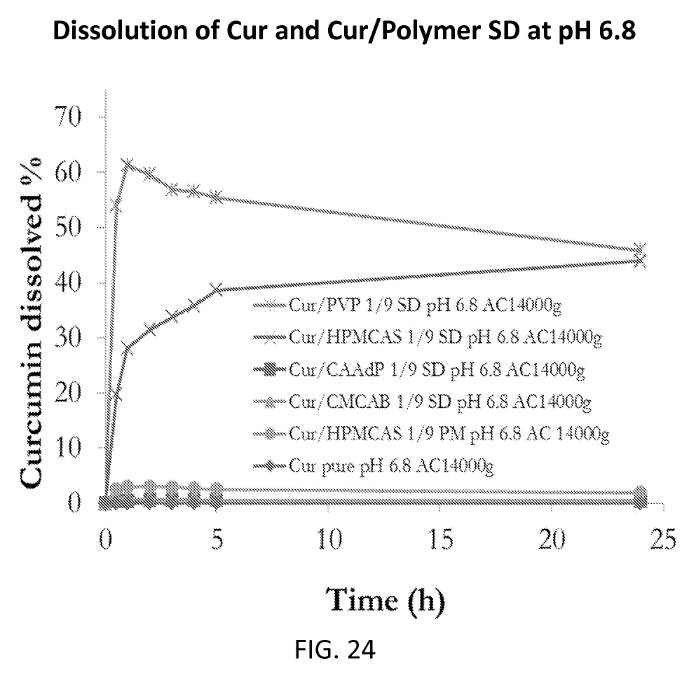

In order to study the effect of different polymer matrices, drug release profiles at pH 6.8 from Cur/PVP 1/9, Cur/HPMCAS 1/9, Cur/CMCAB 1/9 and Cur/CAAdP 1/9 solid dispersions after centrifugation at 14000 g for 10 min were compared (FIG. 24) with pure curcumin and a physical mixture (Cur/HPMCAS 1/9). Release from the Cur/PVP 1/9 solid dispersion was fastest and most complete, reaching 61% within 1 h, while release from the Cur/HPMCAS 1/9 SD blend reached 44% within 4 h. Release from the hydrophobic CMCAB or CAAdP blends (1/9 SD) was quite slow and incomplete, reaching a maximum of only 0.6-0.7% after 3 h. Pure Cur release concentration was below the UV/Vis detection limit. Release from the Cur/HPMCAS 1/9 physical mixture was much slower and less complete than that from the amorphous solid dispersion, reaching only 3.1% after 24 h. Clearly both PVP and HPMCAS are highly effective at suppressing Cur crystallization in both the solid and solution phases, leading to practical levels of Cur release within physiologically relevant time windows.

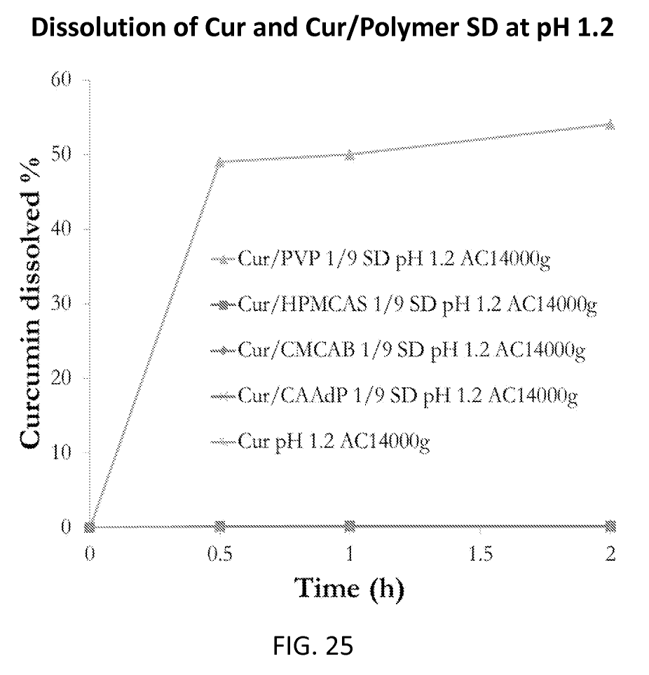

It is also of interest to compare Cur release from these solid dispersions under conditions similar to those of the stomach, in pH 1.2 buffer (FIG. 25). Release from the PVP amorphous blend (Cur/PVP 1/9 SD blend) was substantial, reaching 54% Cur release within 2 h. In contrast, virtually no dissolution was observed from pure curcumin or its amorphous blends with CMCAB, CAAdP, or HPMCAS. Since PVP contains a slightly basic amide group in contrast to the pendent carboxyls of the cellulose esters, this result is exactly what one would expect chemically. Further, it can be expected that CMCAB, CAAdP and HPMCAS would protect curcumin from the stomach contents much more effectively than would PVP.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. It is intended that the specification and examples be considered as exemplary in nature and that variations that do not depart from the essence of the invention are intended to be within the scope of the invention. Further, the references cited in this disclosure are incorporated by reference herein in their entireties.

1. A composition comprising a molecular dispersion of an amorphous solid compound in an amorphous polymer matrix which has increased solubility, stability, or bioavailability over a non-amorphous form of the compound.

2. The composition of claim 1, wherein the non-amorphous form of the compound is a Class II or Class IV drug according to the Biopharmaceutics Classification System in that it exhibits respectively high intestinal permeability but low solubility or low intestinal permeability and low solubility.

3. The composition of claim 1, wherein the amorphous solid is chosen from at least one of curcumin, resveratrol, ellagic acid, naringenin, and quercetin.

4. The composition of claim 1, wherein the amorphous polymer matrix is chosen from at least one of CA adipate, CAB adipate, CAP suberate, CAP sebacate, CAB suberate, CAB sebacate, CA suberate, CA sebacate, HPMCAS (hydroxypropylmethyl cellulose acetate succinate), CAPH (cellulose acetate phthalate), HPMCPH (hydroxypropylmethylcellulose phthalate), CAAdP (cellulose adipate ester), CMCAB (carboxymethylcellulose acetate butyrate), and PVP (polyvinylpyrrolidone).

5. The composition of claim 1, wherein the amorphous polymer matrix is chosen from at least one of carboxylated cellulose esters.

6. The composition of claim 5, wherein the carboxylated cellulose esters are chosen from cellulose acetate adipate propionate, cellulose acetate adipate butyrate, cellulose acetate adipate, cellulose acetate propionate suberate, cellulose acetate propionate sebacate, carboxymethylcellulose acetate butyrate, carboxymethylcellulose acetate propionate, and hydroxymethylcellulose acetate succinate.

7. The composition of claim 1, wherein the amorphous solid compound and the amorphous polymer matrix are combined in a ratio of from about 1:10 to 10:1.

8. The composition of claim 1, wherein a ratio of the amorphous solid compound to the amorphous polymer matrix is about 1:9, 1:3, 1:1, 3:1, or 9:1.

9. A pharmaceutical formulation comprising an amorphous solid form of a Class II or Class IV drug according to the Biopharmaceutics Classification System in that it exhibits respectively high intestinal permeability but low solubility or low intestinal permeability and low solubility, wherein the amorphous solid is present in a molecular dispersion with an amorphous polymer matrix and the formulation has increased solubility, stability, or bioavailability over the Class II or Class IV drug.

10. The pharmaceutical formulation of claim 9, wherein the drug is a flavonoid chosen from curcumin, resveratrol, ellagic acid, naringenin, and quercetin.

11. The pharmaceutical formulation of claim 9, wherein the amorphous polymer matrix is chosen from at least one of CA adipate, CAB adipate, CAP suberate, CAP sebacate, CAB suberate, CAB sebacate, CA suberate, CA sebacate, HPMCAS (hydroxypropylmethyl cellulose acetate succinate), CAPH (cellulose acetate phthalate), HPMCPH (hydroxypropylmethylcellulose phthalate), CAAdP (cellulose adipate ester), CMCAB (carboxymethylcellulose acetate butyrate), and PVP (polyvinylpyrrolidone).

12. The pharmaceutical formulation of claim 9, wherein the amorphous polymer matrix is chosen from at least one of carboxylated cellulose esters.

13. The pharmaceutical formulation of claim 12, wherein the carboxylated cellulose esters are chosen from cellulose acetate adipate propionate, cellulose acetate adipate butyrate, cellulose acetate adipate, cellulose acetate propionate suberate, cellulose acetate propionate sebacate, carboxymethylcellulose acetate butyrate, carboxymethylcellulose acetate propionate, and hydroxymethylcellulose acetate succinate.

14. The formulation of claim 9, wherein the amorphous solid compound and the amorphous polymer matrix are combined in a ratio of from about 1:10 to 10:1.

15. The formulation of claim 9, wherein a ratio of the amorphous solid compound to the amorphous polymer matrix is about 1:9, 1:3, 1:1, 3:1, or 9:1.

16. A method of treating or ameliorating a disease by providing cardioprotective, antioxidant, or anticancer effects by administering to a patient in need thereof a pharmaceutical formulation comprising a flavonoid chosen from curcumin, resveratrol, ellagic acid, naringenin, and quercetin in a polymer matrix.

17. A method for the preparation of a medicament for treating or ameliorating a disease responsive to cardioprotective, antioxidant, or anticancer agents, wherein the medicament comprises a flavonoid chosen from curcumin, resveratrol, ellagic acid, naringenin, and quercetin in a polymer matrix.

18. The pharmaceutical formulation of claim 9, wherein the drug is a bioactive flavonoid miscible with at least one of CA adipate, CAB adipate, CAP suberate, CAP sebacate, CAB suberate, CAB sebacate, CA suberate, CA sebacate, HPMCAS (hydroxypropylmethyl cellulose acetate succinate), CAPH (cellulose acetate phthalate), HPMCPH (hydroxypropylmethylcellulose phthalate), CAMP (cellulose adipate ester), CMCAB (carboxymethylcellulose acetate butyrate), and PVP (polyvinylpyrrolidone).

19. The composition of claim 1 comprising ellagic acid in CMCAB, wherein the ellagic acid is added to CMCAB which is first dissolved in a solvent.
